(19) United States
(12) Patent Application Publication
(10) Pub. No.: US 2024/0099847 A1
(43) Pub. Date: Mar. 28, 2024

(54) SYSTEMS, APPARATUSES, AND METHODS FOR ACCOMMODATING VENTRICULAR EXPANSION

(71) Applicant: EDWARDS LIFESCIENCES CORPORATION, Irvine, CA (US)

(72) Inventors: Jyoti B. Rao, Brea, CA (US); Alyssa E. Kornswiet, Irvine, CA (US)

(21) Appl. No.: 18/527,867

(22) Filed: Dec. 4, 2023

Related U.S. Application Data
(63) Continuation of application No. PCT/US2022/032062, filed on Jun. 3, 2022.
(60) Provisional application No. 63/197,109, filed on Jun. 4, 2021.

(51) Int. Cl. A61F 2/24

(52) U.S. Cl. CPC A61F 2/2487; A61F 2210/0057; A61F 2220/0008; A61F 2220/0025; A61F 2250/0007; A61F 2250/0012

(57) ABSTRACT

Systems, apparatuses, and methods disclosed herein are provided for medical treatment, including treatment of dilated hearts (e.g., dilated left ventricle) or functional mitral valve regurgitation within a human heart. In examples, transcatheter medical treatments may be utilized. The portion of the patient's heart may be dilated due to a myocardial infarction or other cardiomyopathy. The treatment may comprise beating-heart repair of left ventricles with ischemic or non-ischemic dilated cardiomyopathy. The treatments may include approximating papillary muscles of the heart and accommodating ventricular expansion of the heart.

### CROSS-REFERENCE TO RELATED APPLICATIONS

This application is a continuation of International Application No. PCT/US2022/032062, filed Jun. 3, 2022, which claims the benefit of U.S. Patent Application No. 63/197,109, filed Jun. 4, 2021, the entire contents all of which are incorporated by reference for all purposes.

### BACKGROUND

Heart failure can occur when the left ventricle of the heart becomes enlarged and dilated as a result of one or more of various etiologies. Initial causes of heart failure can include chronic hypertension, myocardial infarction, mitral valve incompetency, and other dilated cardiomyopathies. With each of these conditions, the heart is forced to overexert itself in order to provide a cardiac output demanded by the body during various demand states. The result can be an enlarged left ventricle.

A dilated or enlarged heart, and particularly a dilated or enlarged left ventricle, can significantly increase tension and stress in heart walls both during diastolic filling and systolic contraction, which contributes to further dilatation or enlargement of chambers of the heart. In addition, mitral valve incompetency or mitral valve regurgitation is a common comorbidity of congestive heart failure. As the dilation of the ventricle increases, valve function generally worsens, which results in a volume overload condition. The volume overload condition further increases ventricular wall stress, thereby advancing the dilation process, which further worsens valve dysfunction.

In heart failure, the size of the valve annulus (particularly the mitral valve annulus) increases while the area of the leaflets of the valve remains constant. This may lead to reduced coaptation area between the valve leaflets, and, as a result, eventually to valve leakage or regurgitation. Moreover, in normal hearts, the annular size contracts during systole, aiding in valve coaptation. In heart failure, there is poor ventricular function and elevated wall stress. These conditions tend to reduce annular contraction and distort annular size, often exacerbating mitral valve regurgitation. In addition, as the chamber dilates, the papillary muscles (to which the leaflets are connected via the chordae tendineae) may move radially outward and downward relative to the valve, and relative to their normal positions. During this movement of the papillary muscles, however, the various chordae lengths remain substantially constant, which limits the full closure ability of the leaflets by exerting tension prematurely on the leaflets. This condition is commonly referred to as “chordal tethering.” The combination of annular changes and papillary changes results in a poorly functioning valve.

### SUMMARY

Systems, apparatuses, and methods disclosed herein are provided for medical treatment, including treatment of dilated hearts (e.g., dilated left ventricle) or functional mitral valve regurgitation within a human heart. In examples, transcatheter medical treatments may be utilized. The portion of the patient's heart may be dilated due to a myocardial infarction or other cardiomyopathy. The treatment may comprise beating-heart repair of left ventricles with ischemic or non-ischemic dilated cardiomyopathy. The treatments may include approximating papillary muscles of the heart.

The systems, apparatuses, and methods disclosed herein may include applying one or more heart splints to the patient's heart to apply pressure to the heart to approximate the papillary muscles. The heart splints may include anchors connected by a tension member that is tensioned to apply pressure to the patient's heart. The anchors may be positioned in desired locations to approximate the papillary muscles and reshape the heart at particular locations.

In examples herein, the tension member may be configured to expand to accommodate expansion of the ventricle. The tension member may expand to allow for expansion of the ventricle during a diastolic phase, to allow the ventricle to fill with blood. The expansion of the tension member may allow the ventricle to fill more completely with fluid compared to an example in which the tension member does not expand. During a systolic phase, the tension member may return to its original position to allow the heart to contract and eject blood from the ventricle.

In certain examples, the systems, apparatuses, and methods disclosed herein may be utilized in a minimally invasive procedure, to access the heart and apply the heart splint without requiring a full sternotomy.

Any or all of the treatment methods, operations, or steps described herein may be performed on a living human or non-human subject, or on a human or non-human cadaver or portion(s) thereof (e.g., heart, body part, tissue, etc.), simulator, or anthropomorphic ghost, for example, for educational or training purposes.

A system of the present disclosure may be for approximating papillary muscles of a ventricle of a heart. The system may include a first heart anchor configured to couple to a first papillary muscle of the ventricle. The system may include a second heart anchor configured to couple to a second papillary muscle of the ventricle. The system may include a tension member configured to extend within the ventricle and couple the first heart anchor to the second heart anchor and apply a tension to approximate the first papillary muscle and the second papillary muscle, the tension member being configured to expand to accommodate expansion of the ventricle.

A system of the present disclosure may be for approximating papillary muscles of a ventricle of a heart. The system may include a first heart anchor configured to be positioned on a first portion of the heart. The system may include a second heart anchor configured to be positioned on a second portion of the heart. The system may include a tension member configured to extend within the ventricle and couple the first heart anchor to the second heart anchor and apply a tension to approximate the papillary muscles of the ventricle, the tension member being configured to expand to a defined limit to accommodate expansion of the ventricle.

A method of the present disclosure may be for approximating papillary muscles of a ventricle of a heart. The method may include deploying a first heart anchor to a first papillary muscle of the ventricle. The method may include deploying a second heart anchor to a second papillary muscle of the ventricle. The method may include tensioning a tension member for coupling the first heart anchor to the second heart anchor to approximate the papillary muscles of the ventricle, the tension member extending within the ventricle and configured to expand to accommodate expansion of the ventricle.

A method of the present disclosure may be for approximating papillary muscles of a ventricle of a heart. The method may include deploying a first heart anchor to a first portion of the heart. The method may include deploying a second heart anchor to a second portion of the heart. The method may include tensioning a tension member for coupling the first heart anchor to the second heart anchor to approximate the papillary muscles of the ventricle, the tension member extending within the ventricle and configured to expand to a defined limit to accommodate expansion of the ventricle.

### BRIEF DESCRIPTION OF THE DRAWINGS

Features and advantages of the systems, apparatuses, and methods as disclosed herein will become appreciated as the same become better understood with reference to the specification, claims, and appended drawings wherein:

FIG. 1A illustrates a cross sectional view of a portion of a heart.

FIG. 1B illustrates a cross sectional view of a portion of a dilated heart suffering from functional mitral valve regurgitation.

FIG. 2 illustrates a cross sectional view of a portion of a heart showing a heart splint coupled to papillary muscles, according to an example of the present disclosure.

FIG. 3 illustrates a schematic view of a tension member expanding according to an example of the present disclosure.

FIG. 4A illustrates a side view of a tension member in an unexpanded configuration according to an example of the present disclosure.

FIG. 4B illustrates a side view of the tension member shown in FIG. 4A in an expanded configuration according to an example of the present disclosure.

FIG. 5A illustrates a side view of a tension member in an unexpanded configuration according to an example of the present disclosure.

FIG. 5B illustrates a side view of the tension member shown in FIG. 5A in an expanded configuration according to an example of the present disclosure.

FIG. 6A illustrates a side view of a tension member in an unexpanded configuration according to an example of the present disclosure.

FIG. 6B illustrates a side view of the tension member shown in FIG. 6A in an expanded configuration according to an example of the present disclosure.

FIG. 7A illustrates a side view of a tension member in an unexpanded configuration according to an example of the present disclosure.

FIG. 7B illustrates a cross sectional view of the tension member shown in FIG. 7A in an unexpanded configuration according to an example of the present disclosure.

FIG. 7C illustrates a side view of the tension member shown in FIG. 7A in an expanded configuration according to an example of the present disclosure.

FIG. 8 illustrates a cross sectional view of a heart splint coupled to papillary muscles according to an example of the present disclosure.

FIG. 9 illustrates a cross sectional view of a heart splint coupled to papillary muscles according to an example of the present disclosure.

FIG. 10 illustrates a cross sectional view of a heart splint coupled to papillary muscles according to an example of the present disclosure.

FIG. 11 illustrates a cross sectional view of the heart splint shown in FIG. 10 in an expanded configuration and coupled to papillary muscles according to an example of the present disclosure.

FIG. 12 illustrates a cross sectional view of a heart splint coupled to papillary muscles according to an example of the present disclosure.

FIG. 13 illustrates a perspective view of a tension member according to an example of the present disclosure.

FIG. 14 illustrates a perspective view of the tension member shown in FIG. 13 in an expanded configuration according to an example of the present disclosure.

FIG. 15 illustrates a cross sectional schematic view of the tension member shown in FIG. 13 in an unexpanded configuration.

FIG. 16 illustrates a cross sectional schematic view of the tension member shown in FIG. 13 in an expanded configuration.

FIG. 17 illustrates a cross sectional view of a heart splint applied to a patient's heart according to an example of the present disclosure.

FIG. 18A illustrates a front view of a ring of a heart anchor according to an example of the present disclosure.

FIG. 18B illustrates a side view of the ring of a heart anchor shown in FIG. 18A.

FIG. 18C illustrates a top view of an unfolded cover of a heart anchor according to an example of the present disclosure.

FIG. 18D illustrates a top view of the cover shown in FIG. 18C folded.

FIG. 18E illustrates a side view of the folded cover shown in FIG. 18D.

FIG. 18F illustrates an alternate configuration of a cover of a heart anchor according to an example of the present disclosure.

FIG. 18G illustrates a heart anchor according to an example of the present disclosure.

FIG. 18H illustrates the heart anchor shown in FIG. 18G in a linearized configuration.

FIG. 19A illustrates a side view of a deployment apparatus with an anchor partially extending out of a deployment apparatus according to an example of the present disclosure.

FIG. 19B illustrates a side view of a deployment apparatus with an anchor extending out of a deployment apparatus according to an example of the present disclosure.

FIG. 20A illustrates a cross sectional view of a heart anchor according to an example of the present disclosure.

FIG. 20B illustrates a cross sectional view of the heart anchor shown in FIG. 20A according to an example of the present disclosure.

### DETAILED DESCRIPTION

Various aspects of the present disclosure generally relate to systems, apparatuses, and methods for medical treatment and/or treating heart conditions, including, by way of example, treating dilation/dilatation (including a dilated left ventricle), valve incompetencies (including mitral valve regurgitation), and other similar heart conditions. The systems, apparatuses, and methods in examples may be adapted for transcatheter medical treatments that may not require full, open surgery, and can be minimally invasive. The systems, apparatus, and methods may be utilized to approximate one or more papillary muscles of a patient's heart, and may reshape the left ventricle in examples.

In certain examples, the present disclosure involves geometric reshaping of the heart and treating valve incompetencies. In certain aspects of the present disclosure, the papillary muscles may be approximated to reduce chordal tethering, to relieve functional mitral regurgitation. In examples, the systems, apparatuses, and methods may be utilized to approximate papillary muscles of the right ventricle, to relive functional tricuspid regurgitation.

In examples, the systems, apparatuses, and methods disclosed herein may be utilized in a beating heart procedure. Such a procedure may involve deployment of the heart splint while the heart is beating. In such an example, the papillary muscle approximation amount may be fine tuned while monitoring hemodynamics in real-time and locking the heart splint in position when a desired result is reached. Rapid pacing may be utilized at desired times to minimize heart motion and improve ease of targeting desired puncture locations on the heart.

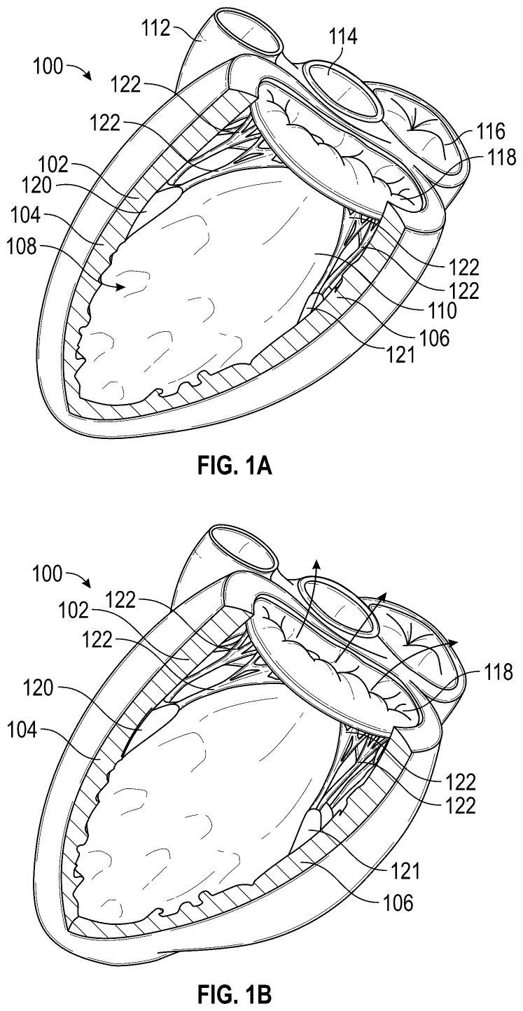

FIG. 1A illustrates an example of a heart 100, with a partial cross sectional view of the left ventricle 102. An anterior wall 104 of the left ventricle 102 is shown as well as a posterior wall 106 of the left ventricle 102. The interior cavity 108 of the left ventricle 102, and the interventricular septum no are shown. The pulmonary artery 112, aorta 114, and tricuspid valve 116 are also shown in a representative view.

The mitral valve 118 is shown in FIG. 1A. The heart 100 shown in FIG. 1A represents a normally operating heart, in which the leaflets of the mitral valve 118 coapt properly and the annulus of the mitral valve 118 has a shape allowing the leaflets of the mitral valve 118 to coapt properly. The papillary muscles 120, 121 are positioned relative to each other and relative to the mitral valve 118 such that the chordae 122 extending to the mitral valve leaflets properly tether to the leaflets and allow the leaflets to close and open in a desired manner.

The heart 100 may suffer from maladies that alter the position of the papillary muscles 120, 121. Such maladies may comprise enlargement of the left ventricle 102. Such enlargement may be caused by ischemic or non-ischemic dilated cardiomyopathy, among other maladies. Abnormal tethering forces on the chordae 122 may result from displacement of the papillary muscles 120, 121 due to enlargement of the left ventricle 102.

FIG. 1B, for example, illustrates the heart 100 with a dilated left ventricle 102. The papillary muscles 120, 121 have moved away from each other thus producing abnormal tethering forces upon the chordae 122. The leaflets of the mitral valve 118 may not coapt properly, thus leading to functional mitral regurgitation as represented by the arrows in FIG. 1B.

Treatment for such a condition may comprise approximating the papillary muscles 120, 121 to reduce the tension upon the chordae 122 and thus allowing the leaflets to return closer to a native state of coaptation. A heart splint may be utilized to approximate the papillary muscles 120, 121. The heart splint may include heart anchors and a tension member configured to couple the heart anchors to each other and extend within a ventricle of the heart.

In methods of approximating the papillary muscles in which a non-expandable tension member is utilized, the papillary muscles may be restricted from movement during diastolic filling of the ventricle due to the tension provided by such a tension member. Such lack of movement may be undesirable because it may reduce the ability of the ventricle to fill completely during diastole and may produce additional subvalvular dysfunction.

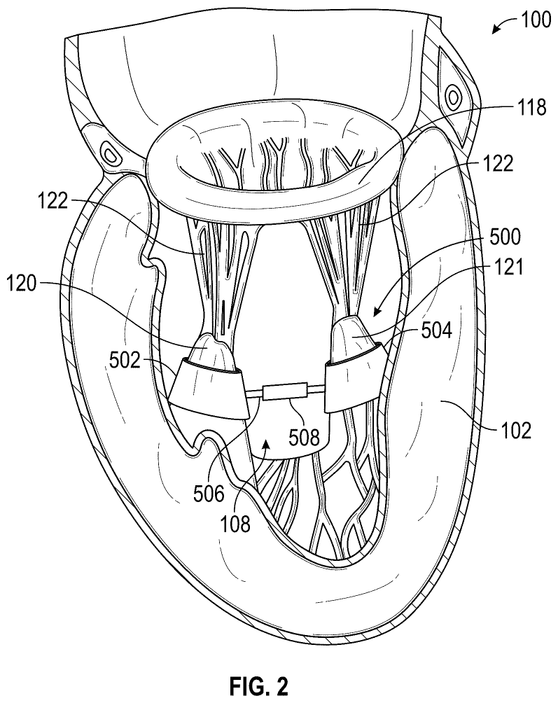

FIG. 2 illustrates a heart splint 500 that may be utilized in examples herein. The heart splint 500 may include a first heart anchor 502 configured to be positioned on a first portion of the heart 100, a second heart anchor 504 configured to be positioned on a second portion of the heart 100, and a tension member 506.

The first heart anchor 502 may be configured to couple to a papillary muscle 120 of the ventricle 102, to anchor the heart splint 500 to the papillary muscle 120. In examples, as shown in FIG. 2, the first heart anchor 502 may comprise a cuff that extends around the papillary muscle 120. The first heart anchor 502 may have other configurations as desired, such as one or more sutures or another form of anchor as desired in examples.

The second heart anchor 504 may be configured to couple to another papillary muscle 121 of the ventricle 102, to anchor the heart splint 500 to the papillary muscle 121. In examples, as shown in FIG. 2, the second heart anchor 504 may comprise a cuff that extends around the papillary muscle 121. The second heart anchor 504 may have other configurations as desired, such as one or more sutures or another form of anchor as desired in examples.

In examples, the first heart anchor 502 and second heart anchor 504 may couple to the respective papillary muscles 120, 121 to anchor and secure to the papillary muscles 120, 121 for approximation of the papillary muscles 120, 121. In examples, a first heart anchor and a second heart anchor may be coupled to other portions of the heart, including on one or more ventricular walls or other portions of the heart, for approximation of the papillary muscles 120, 121.

The tension member 506 may be configured to extend within the ventricle and couple the first heart anchor 502 to the second heart anchor 504 and apply a tension to approximate the papillary muscles 120, 121 of the ventricle 102. The tension member 506, for example, may comprise a tether that is configured to apply a tension to the first heart anchor 502 and the second heart anchor 504, to draw such anchors 502, 504 towards each other to correspondingly approximate the papillary muscles 120, 121. Such approximation may have the benefit of reducing chordal tethering, reducing a size of the ventricle 102, and/or addressing functional mitral valve regurgitation. The tension member 506 may include one or more of a cord, a wire, a chain, or other form of tension member 506 configured to couple to the first heart anchor 502 and the second heart anchor 504.

In examples, the tension member 506 may be configured to expand to accommodate expansion of the ventricle 102. The expansion may comprise a longitudinal stretching of the tension member 506 in examples. Such a feature may be a property of the materials of the tension member 506, or in examples the tension member 506 may include an expandable body 508 that may allow the tension member 506 to expand. For example, the expandable body 508 may be configured to move from an unexpanded configuration to an expanded configuration to allow the tension member 506 to move from an unexpanded configuration to an expanded configuration. The unexpanded configuration may be an unstretched configuration in examples and the expanded configuration may be a stretched configuration in examples. The unexpanded configuration of the tension member 506 may correspond to a systolic phase of the ventricle 102, and the expanded configuration of the tension member 506 may correspond to a diastolic phase of the ventricle 102, for example. As such, the tension member 506 may be configured to expand to allow the papillary muscles 120, 121 to move during the cardiac cycle, to allow for improved filling of the ventricle 102, among other benefits.

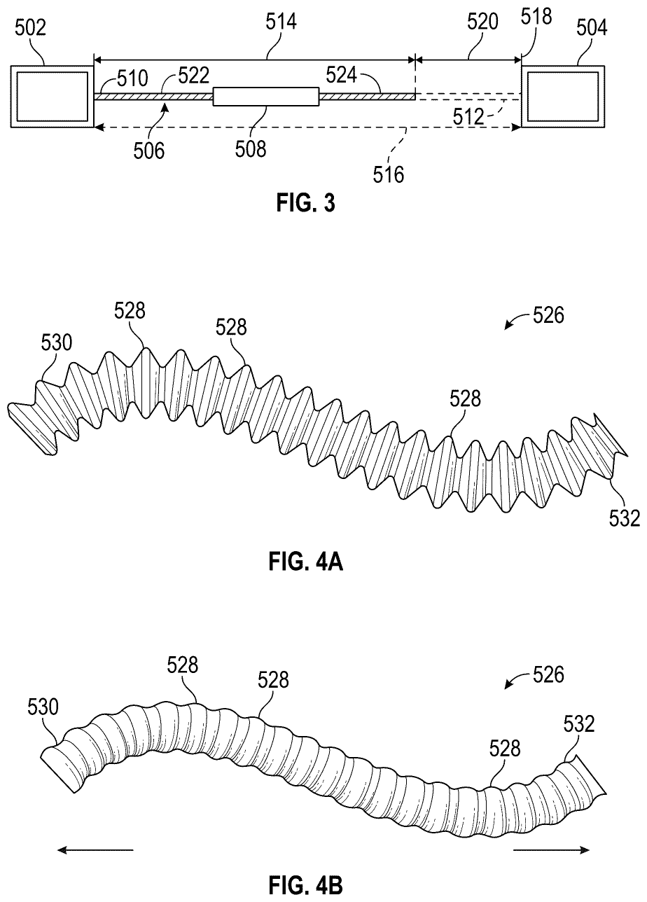

FIG. 3 illustrates a schematic representation of an operation of the tension member 506. The position of the tension member 506 in an unexpanded configuration is shown in solid lines, and the position of the tension member 506 in an expanded configuration is shown in dashed lines.

The tension member 506 has a first end portion 510 configured to couple to the first heart anchor 502 and a second end portion 512 configured to couple to the second heart anchor 504. The tension member 506 may have a length between the first heart anchor 502 and the second heart anchor 504. The length may vary due to the tension member 506 expanding or stretching longitudinally.

FIG. 3, for example, illustrates the tension member 506 having a length 514 in the unexpanded configuration. The length 514 may correspond to a length between the first heart anchor 502 and the second heart anchor 504 during a systolic phase of the ventricle 102. The tension member 506 may be configured to expand to a greater second length 516. The second length 516 may correspond to a length between the first heart anchor 502 and the second heart anchor 504 during a diastolic phase of the ventricle 102.

The tension member 506 may be configured to expand to a defined limit to accommodate expansion of the ventricle 102 in examples. As such, the tension member 506 may be configured to expand to the second length 516 and then expand no further beyond the second length 516 in examples. Such a configuration may allow the tension member 506 to retain a compressive force to the papillary muscles 120, 121 even in a diastolic phase. Thus, as the ventricle 102 expands during a diastolic phase, the increase in the distance between the papillary muscles 120, 121 may remain limited by the defined limit (as marked by line 518 in FIG. 3) that the tension member 506 may expand to. The tension member 506 may apply a defined compressive force even in a diastolic phase in which the ventricle 102 has expanded. As such, in examples, the tension member 506 may differ from an expandable tension member that might instead expand for an undefined length.

The tension member 506 may be configured to be elastic in examples, and may apply an elastic force at the second length 516, and/or the length 520 that is between the first length 514 and the second length 516. The tension member 506 may be configured to expand or stretch between a first length 514 at a systolic phase of the ventricle 102 and a second length 516 at a diastolic phase of the ventricle 102, and apply an elastic force between the first length 514 and the second length 516. In examples, the tension member 506 may be configured to apply an elastic force at the first length 514.

An elastic force applied by the tension member 506 may serve to approximate the first heart anchor 502 and the second heart anchor 504 and accordingly the papillary muscles 120, 121. An elastic force applied at the second length 516 and/or the length 520 may serve to return the tension member 506 to the first length 514 upon the ventricle 102 returning to the systolic phase during the cardiac cycle, and may aid to approximate the papillary muscles 120, 121 at the second length 516 and/or the length 520.

An elastic force applied by the tension member 506 may have a strength that allows the ventricle 102 to expand during diastolic filling. Thus, the strength of the ventricle 102 may be sufficient to expand the tension member 506 to the second length 516. However, in examples, the defined limit of expansion of the tension member 506 may be sufficient to stop any further increase in the distance between the papillary muscles 120, 121, or may stop the tension member 506 from allowing the ventricle 102 to continue to expand to an undesired dilated state as shown in FIG. 1B for example. The tension member 506 may be configured to allow the heart to relax during a filling phase while still providing a restriction to treat functional mitral regurgitation, for example.

In examples, the entirety of the tension member 506 may be configured to expand, or at least a portion of the tension member 506 may be configured to expand. For example, the tension member 506 may include one or more portions 522, 524 that comprise tethers that are non-expandable or non-stretchable. Such tethers may be in the form of a cord, a wire, or a chain, or other forms of tethers that may not expand. The tension member 506 however may include an expandable body 508 that is configured to expand and provides expansion properties of the tension member 506 in examples. The portions 522, 524 may be positioned adjacent to the expandable body 508 in examples if desired. In examples, the entirety of the tension member 506 may comprise an expandable body as disclosed herein.

FIG. 4A, for example, illustrates an example of an expandable body 526 that may be utilized in examples herein. The expandable body 526 may correspond to the expandable body 508 shown in regard to FIG. 3, or in examples the entirety of the tension member 506 may comprise the expandable body 526. The expandable body 526 is shown in an unexpanded configuration in FIG. 4A (corresponding, for example to the length 514 shown in FIG. 3).

The expandable body 526 includes corrugations 528 that may be configured to allow the tension member 506 to expand. The corrugations 528 for example, may allow the expandable body 526 to have portions that fold when the tension member 506 is at the first length 514 shown in FIG. 3. The corrugations 528 may further be configured to provide an elastic force that draws the tension member 506 to the first length 514. The material properties of the expandable body 526, for example, may be configured to retain the corrugations 528 in the configuration shown in FIG. 4A, and an expansion or stretching force applied by the ventricle 102 may expand the expandable body 526 so that the tension member 506 reaches a second length 516 (as marked in FIG. 3). The expandable body 526 may be biased to the unstretched configuration shown in FIG. 4A, however, and may provide an elastic force to return the expandable body 526 to the configuration shown in FIG. 4A.

FIG. 4B illustrates the expandable body 526 in an expanded configuration (corresponding, for example to the length 516 shown in FIG. 3). The expandable body 526 has expanded to correspondingly expand the tension member 506 to the expanded configuration. The corrugations 528 flatten and unfold upon the expandable body 526 expanding. An outer diameter of at least a portion of the tension member 506 accordingly decreases upon the tension member expanding.

The expandable body 526 may be configured to expand to a defined limit. As such, the expandable body 526 may expand to a length and then expand no further, to continue to apply a compressive force against the ventricle 102. The limit, for example, may comprise a configuration in which the corrugations 528 are entirely flattened, although other limits may be provided as desired (e.g., the corrugations 528 partially flattening to a certain extent). In examples, a configuration of the corrugations 528, such as a depth or width of the corrugations 528 may be set to define the limit that the expandable body 526 may expand to. In examples, material properties of the expandable body 526 may be set to define the limit that the expandable body 526 may expand to. In examples, the configuration of the expandable body 526 may be set to define a force that may expand the expandable body 526 (e.g., the force of ventricular expansion), and define an elastic force that may be applied by the expandable body 526 to return to the unexpanded configuration.

The expandable body 526 may iteratively cycle between the unexpanded configuration shown in FIG. 4A and the expanded configuration shown in FIG. 4B during the cardiac cycle.

The expandable body 526 may comprise a hose that may have the corrugations 528. The expandable body 526 may have a smooth outer surface that may reduce the possibility of snagging or ensnaring with chordae 122, as marked in FIG. 2 for example. In examples, the expandable body 526 may comprise an interwoven strip of a thin, semi-rigid film, or may be made of a plastic, a metal, or a cloth if desired. In examples, other configurations of expandable bodies 526 may be utilized as desired.

The expandable body 526 may be coupled to the portions 522, 524 of the tension member 506 shown in FIG. 3 via one or more couplers. For example, a first end portion 530 of the expandable body 526 may couple to the portion 522 via a coupler, and a second end portion 532 of the expandable body 526 may couple to the portion 524 via a coupler. In examples, the expandable body 526 may be integral with the portions 522, 524 in examples. For example, the expandable body 526 may be formed integrally with the portions 522, 524 and may be formed having the corrugations 528 shown in FIGS. 4A and 4B. In examples, the portions 522, 524 may lack corrugations and may be non-expandable.

In examples, the expandable body 526 may be integral with one or more of the heart anchors 502, 504. For example, the first end portion 530 and second end portion 532 of the expandable body 526 may be integral with respective first and second heart anchors 502, 504. The entirety of the tension member 506 in examples may comprise the expandable body 526. Other configurations of expandable bodies may be utilized in examples.

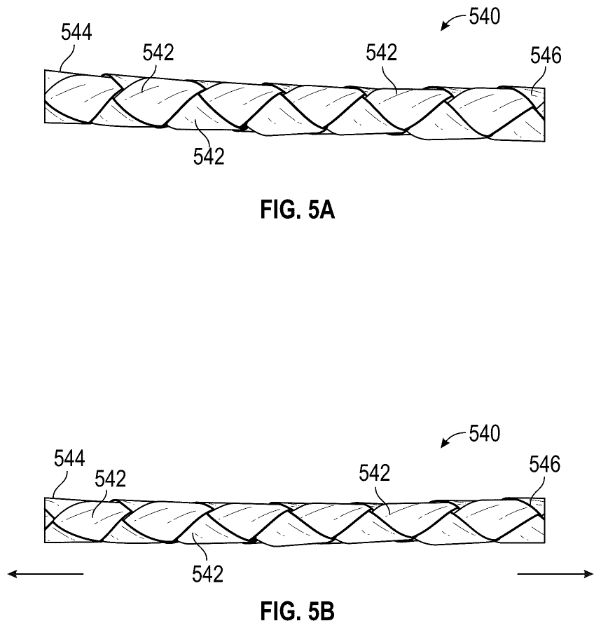

FIG. 5A, for example, illustrates an example of an expandable body 540 that may be utilized in examples herein. The expandable body 540 may correspond to the expandable body 508 shown in regard to FIG. 3, or in examples the entirety of the tension member 506 may comprise the expandable body 540. The expandable body 540 is shown in an unexpanded configuration in FIG. 5A (corresponding, for example to the length 514 shown in FIG. 3).

The expandable body 540 comprises a woven body that may be configured to allow the tension member 506 to expand. For example, the woven body may comprise a plurality of strips 542 that are configured to slide with respect to each other to allow the tension member 506 to expand. The plurality of strips 542 may be configured to slide longitudinally to allow the tension member 506 to expand. At least a portion of the tension member 506 may include the woven body.

The woven body may comprise a tube formed by the weave of the plurality of strips 542. The weave of the plurality of strips 542 may comprise an interleaved cylindrical configuration of the strips 542 and may be configured to provide an elastic force that draws the expandable body 540 to the configuration shown in FIG. 5A. The elastic force may draw the tension member 506 to the first length 514. The weave of the plurality of strips 542, for example, may be configured to retain the woven body in the configuration shown in FIG. 5A, and an expansion force applied by the ventricle 102 may expand the expandable body 540 so that the tension member 506 reaches a second length 516 (as marked in FIG. 3). The weave of the plurality of strips 542 may allow the expandable body 540 to be biased to the unexpanded configuration shown in FIG. 5A, however, and may provide an elastic force to return the expandable body 540 to the configuration shown in FIG. 5A.

FIG. 5B illustrates the expandable body 540 in an expanded configuration (corresponding, for example to the length 516 shown in FIG. 3). The expandable body 540 has expanded to correspondingly expand the tension member 506 to the expanded configuration. The strips 542 slide longitudinally upon the expandable body 540 expanding. An outer diameter of at least a portion of the tension member 506 decreases upon the tension member expanding.

The expandable body 540 may be configured to expand to a defined limit. As such, the expandable body 540 may expand to a length and then expand no further, to continue to apply a compressive force against the ventricle 102. The plurality of strips 542 may be configured to slide longitudinally to a defined limit. The limit, for example, may comprise a configuration in which the strips 542 tighten against each other such that further expansion may not continue. In examples, a configuration of the strips 542, including a weave, a size, or other configuration may be set to define the limit that the expandable body 540 may expand to. In examples, material properties of the expandable body 540 may be set to define the limit that the expandable body 540 may expand to. In examples, the configuration of the strips 542 may be set to define a force that may expand the expandable body 540 (e.g., the force of ventricular expansion), and may define an elastic force that may be applied by the expandable body 540 to return to the unexpanded configuration. In examples, the strips 542 may comprise a thin, semi-rigid film, or may be made of a plastic, a metal, or a cloth if desired.

The expandable body 540 may iteratively cycle between the unexpanded configuration shown in FIG. 5A and the expanded configuration shown in FIG. 5B during the cardiac cycle.

The expandable body 540 may be coupled to the portions 522, 524 of the tension member 506 shown in FIG. 3 via one or more couplers. For example, a first end portion 544 of the expandable body 540 may couple to the portion 522 via a coupler, and a second end portion 546 of the expandable body 540 may couple to the portion 524 via a coupler. In examples, the expandable body 540 may be integral with the portions 522, 524 in examples. For example, the expandable body 540 may be formed integrally with the portions 522, 524.

In examples, the expandable body 540 may be integral with one or more of the heart anchors 502, 504. For example, the first end portions 544 and second end portions 546 of the expandable body 540 may be integral with respective first and second heart anchors 502, 504. The entirety of the tension member 506 in examples may comprise the expandable body 540. Other configurations of expandable bodies may be utilized in examples.

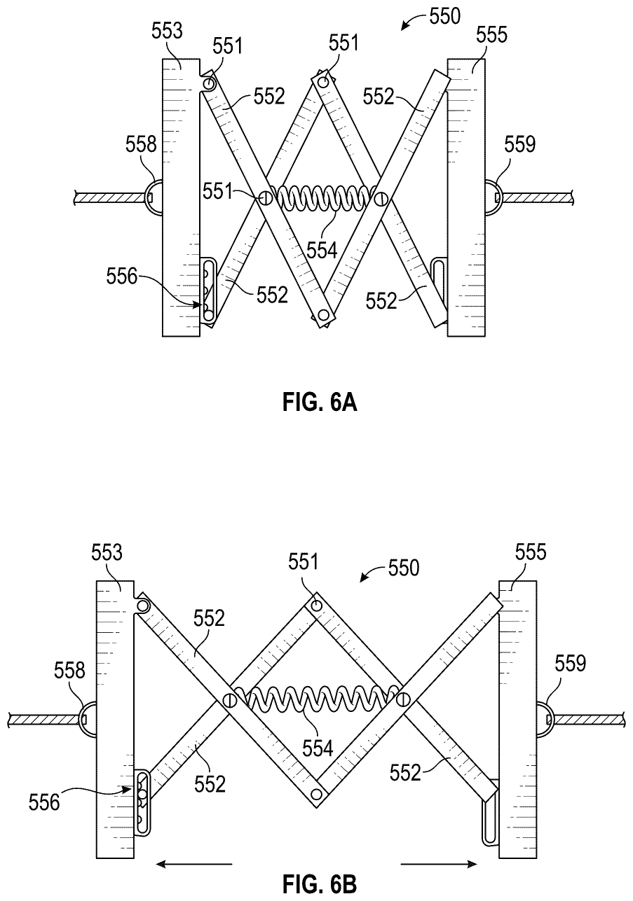

FIG. 6A, for example, illustrates an example of an expandable body 550 that may be utilized in examples herein. The expandable body 550 may correspond to the expandable body 508 shown in regard to FIG. 3, or in examples the entirety of the tension member 506 may comprise the expandable body 550. The expandable body 550 is shown in an unexpanded configuration in FIG. 6A (corresponding to the length 514 shown in FIG. 3 for example).

The expandable body 550 includes a plurality of arms 552 configured to scissor to allow the tension member 506 to expand. For example, the angle between the plurality of arms 552 may vary to allow the expandable body 550 to both increase and decrease in length. The arms 552 may cross each other and be coupled to each other at pivots 551 that may allow the angle between the plurality of arms 552 to vary. The arms 552 may be coupled to pivots at a first end portion 553 and pivots at a second end portion 555, to allow the arms 552 to pivot when the first and second end portions 553, 555 are pulled away from each other and drawn towards each other. The first end portion 553 and second end portion 555 may comprise arms that extend parallel with respect to each other, although other forms of end portions may be utilized in examples. At least a portion of the tension member 506 may include the plurality of arms 552.

The expandable body 550 may include a spring 554 configured to bias the expandable body to the unexpanded configuration. The spring 554 may be coupled to the plurality of arms 552 and may be configured to apply an elastic force to the plurality of arms 552. The spring 554 may draw the tension member 506 to the first length 514 (as marked in FIG. 3). An expansion force applied by the ventricle 102 may expand the expandable body 550 so that the tension member 506 reaches a second length 516 (as marked in FIG. 3). The spring 554 may allow the expandable body 550 to be biased to the unexpanded configuration shown in FIG. 6A and may provide an elastic force to return the expandable body 540 to the configuration shown in FIG. 6A.

The expandable body 550 may expand to a defined limit, defined by a stop 556 that engages a portion of the plurality of arms 552. The stop 556 may be configured to set a defined limit that the expandable body 550 and accordingly the tension member 506 may expand longitudinally to. An end portion of one of the plurality of arms 552 for example may be engaged by the stop 556 to define the length that the expandable body 550 may expand to.

The stop 556 may be adjustable to adjust the defined length that the expandable body 550 may expand to. For example, the end portion of one of the plurality of arms may be placed in a different position with regard to the stop 556 to vary the length that the expandable body 550 may expand to. The stop 556 as show in FIGS. 6A and 6B may comprise a plurality of protrusions that the end portion of one of the plurality of arms 552 may engage and may be selectively positioned with respect to. For example, placing the end portion between different protrusions may vary the defined length that the expandable body 550 may expand to.

FIG. 6B illustrates one of the plurality of arms 552 in a different position with respect to the stop 556 than shown in FIG. 6A, and thus configured to extend to a different limit than possible with the position shown in FIG. 6A.

FIG. 6B illustrates the expandable body 550 in an expanded configuration (corresponding, for example to the length 516 shown in FIG. 3). The expandable body 550 has expanded to correspondingly expand the tension member 506 to the expanded configuration. The end portions 553, 555 have moved away from each other and the arms 552 have scissored to allow the expandable body 550 to expand. An outer diameter of at least a portion of the tension member 506 accordingly decreases upon the tension member expanding.

The expandable body 550 may be configured to expand to the defined limit defined by the stop 556. As such, the expandable body 550 may expand to a length and then expand no further, to continue to apply a compressive force against the ventricle 102. The limit, for example, may comprise a configuration in which the arms 552 can scissor no further or the spring 554 may extend no further. In examples, a configuration of the arms 552, including a size, position, or coupling to each other may be set to define the limit that the expandable body 550 may expand to. The spring 554 may apply an elastic force to return to the configuration shown in FIG. 6A.

The expandable body 550 may iteratively cycle between the unexpanded configuration shown in FIG. 6A and an expanded configuration as shown in FIG. 6B during the cardiac cycle.

The expandable body 550 may be coupled to the portions 522, 524 of the tension member 506 shown in FIG. 3 via one or more couplers. For example, the first end portion 553 of the expandable body 550 may couple to the portion 522 via a coupler 558, and the second end portion 555 may couple to the portion 524 via a coupler 559. In examples, the expandable body 550 may be integral with the portions 522, 524 in examples. For example, the expandable body 550 may be formed integrally with the portions 522, 524.

In examples, the expandable body 550 may be integral with one or more of the heart anchors 502, 504. For example, the first end portion 553 and second end portion 555 of the expandable body 550 may be integral with respective first and second heart anchors 502, 504. The entirety of the tension member 506 in examples may comprise the expandable body 550. Other configurations of expandable bodies may be utilized in examples.

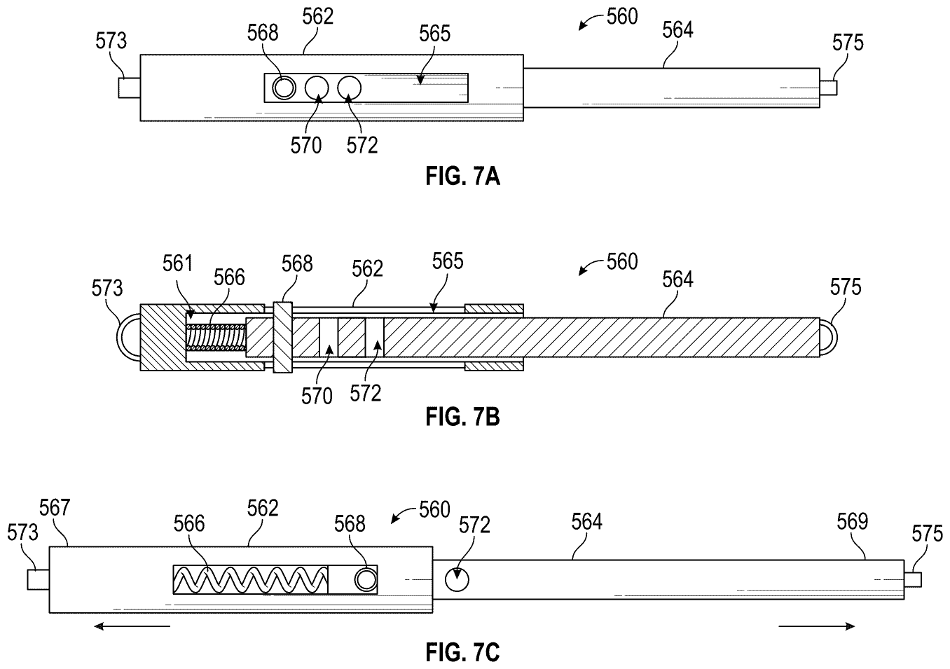

FIG. 7A illustrates an example of an expandable body 560 that may be utilized in examples herein. The expandable body 560 may correspond to the expandable body 508 shown in regard to FIG. 3, or in examples the entirety of the tension member 506 may comprise the expandable body 560. The expandable body 560 is shown in an unexpanded configuration in FIGS. 7A and 7B (corresponding, for example to the length 514 shown in FIG. 3).

The expandable body 560 includes a piston that may be configured to allow the tension member 506 to expand. For example, the piston may include a housing 562 and a plunger 564 configured to slide within the housing 562. At least a portion of the tension member 506 may include the piston.

FIG. 7B illustrates a cross sectional view of the expandable body 560. The piston may include a spring 566 configured to bias the plunger 564 with respect to the housing 562. The spring 566 may be configured to apply an elastic force to the plunger 564. The spring 566 may be positioned within a chamber 561 of the housing 562 as shown in FIG. 7B for example, between an end of the plunger 564 and an interior wall of the housing 562.

The spring 566 may be configured to bias the expandable body 560 to the unexpanded configuration. The spring 566 may draw the tension member 506 to the first length 514 (as marked in FIG. 3). An expansion or stretching force applied by the ventricle 102 may expand the expandable body 560 so that the tension member 506 reaches a second length 516 (as marked in FIG. 3). The spring 566 may allow the expandable body 560 to be biased to the unexpanded configuration shown in FIGS. 7A and 7B and may provide an elastic force to return the expandable body 560 to the configuration shown in FIGS. 7A and 7B.

The piston may include a stop 568 that may be configured to impede movement of the plunger 564 relative to the housing 562. The stop 568 for example, may comprise a pin that protrudes within a channel 565 of the housing 562 and stops movement of the plunger 564 by contacting a wall of the housing 562. As such, the piston may expand no further than the limit defined by the stop 568.

The stop 568 may be adjustable to adjust the defined limit that the piston may expand to. For example, the stop 568 may be repositionable within various openings 570, 572 in the plunger 564. The various openings 570, 572 may be positioned at various lengths along the plunger 564. The stop 568 may be adjustable to vary the defined limit that the expandable body 560 expands to.

FIG. 7C, for example, illustrates the plunger having expanded to a defined limit defined by the position of the stop 568. The expandable body 560 is shown in an expanded configuration (corresponding, for example to the length 516 shown in FIG. 3). The expandable body 560 has expanded to correspondingly expand the tension member 506 to the expanded configuration.

The expandable body 560 may be configured to expand to the defined limit defined by the stop 568. In examples, a configuration of the piston, including a size or position of the components may be set to define the limit that the expandable body 560 may expand to.

The expandable body 560 may iteratively cycle between the unexpanded configuration shown in FIGS. 7A and 7B and the expanded configuration as shown in FIG. 7C during the cardiac cycle.

The expandable body 560 may be coupled to the portions 522, 524 of the tension member 506 shown in FIG. 3 via one or more couplers. For example, a first end portion 567 of the expandable body 560 may couple to the portion 522 via a coupler 573, and the second end portion 569 may couple to the portion 524 via a coupler 575. In examples, the expandable body 560 may be integral with the portions 522, 524 in examples. For example, the expandable body 560 may be formed integrally with the portions 522, 524.

In examples, the expandable body 560 may be integral with one or more of the heart anchors 502, 504. For example, the first end portions 567 and second end portions 569 of the expandable body 550 may be integral with respective first and second heart anchors 502, 504. The entirety of the tension member 506 in examples may comprise the expandable body 560. Other configurations of expandable bodies may be utilized in examples.

Various other configurations of expandable bodies and tension members may be utilized as desired.

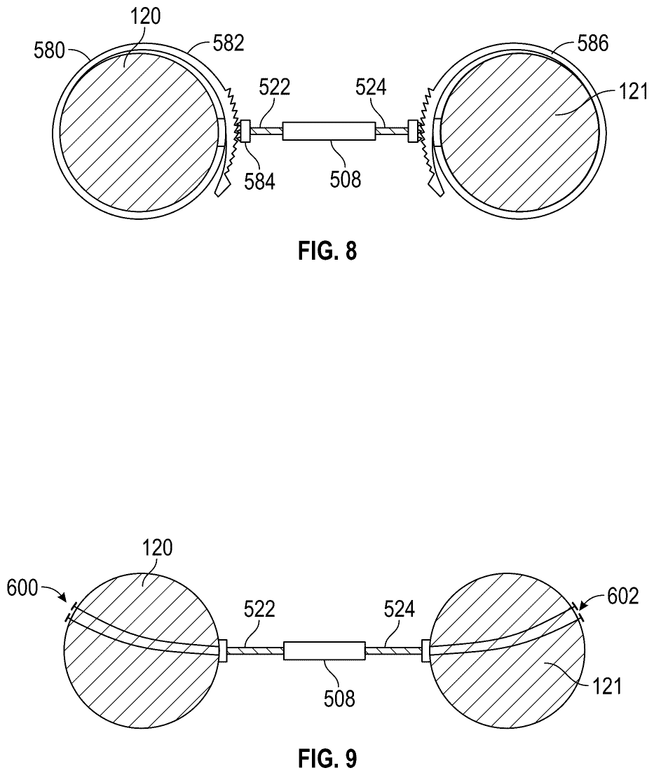

FIG. 8 illustrates a cross sectional view of a configuration of heart anchors that may be utilized according to examples herein. A first heart anchor 580 may comprise a cuff including a strap 582 that extends around a papillary muscle 120 and engages a lock 584 of the heart anchor 580. At least one of the first heart anchor 580 or the second heart anchor 586 may comprise a cuff, or both may comprise a cuff as shown in FIG. 8.

In examples, the strap 582 may include engaging members in the form of teeth that may engage one or more protrusions on the lock 584. The lock 584 in examples may comprise a ratcheting lock that allow for the strap 582 to be tightened yet not loosened without a release of the lock 584. For example, a direction of the teeth on the strap 582 may be configured to allow the strap 582 to be tightened and locked in position automatically upon passing through the ratcheting lock in a direction. The strap 582 may not be loosened unless the lock 584 is released.

The second heart anchor 586 may be configured similarly as the first heart anchor 580 in examples, or may have a different configuration as desired.

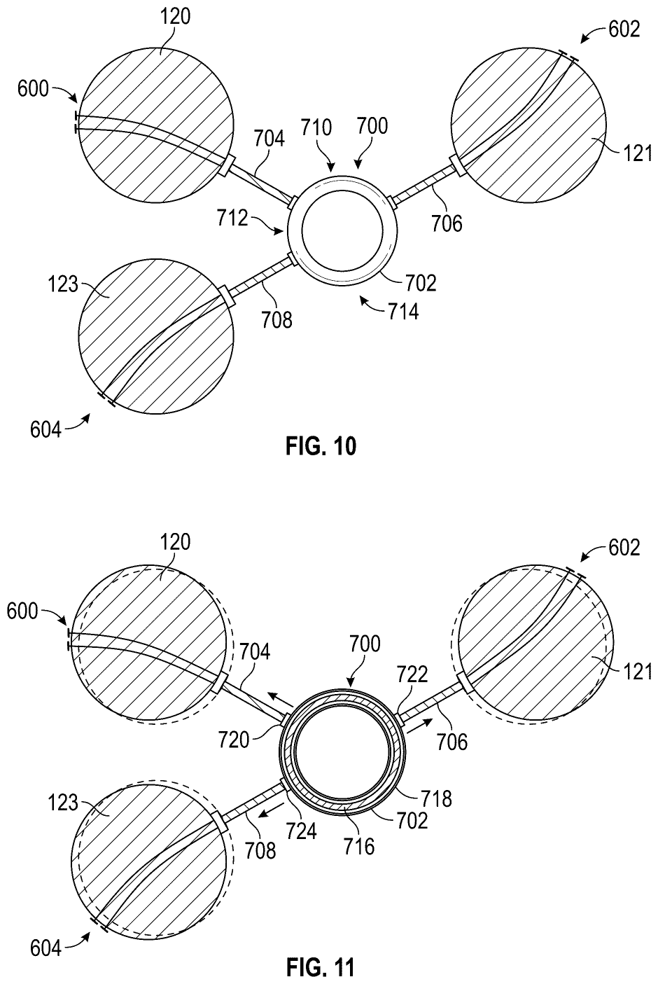

FIG. 9 illustrates an example in which the heart anchors 600, 602 may comprise one or more sutures that are configured to couple to the papillary muscles 120, 121. The sutures, for example, may pass through the papillary muscles 120, 121, as shown in FIG. 9, or may extend around a portion of the papillary muscles 120, 121. At least one of the first heart anchor 600 or the second heart anchor 602 may comprise one or more sutures in examples.

In deployment, the heart anchors may be delivered to the patient's heart in a minimally invasive manner. For example, transcatheter methods may be utilized for deployment if desired. Such transcatheter methods may include passing the heart splint into the patient's body via the vasculature of the patient's body and/or may include passing one or more catheters in a surgical method such as a thoracotomy if desired. The entry into the patient's heart may be via passage through an interventricular septum or interatrial septum, or may be via passage through an outer heart wall such as an outer ventricular wall. Other methods of entry may be provided. In examples, more invasive surgical methods may be utilized to access the heart.

A method of deployment may include deploying a first heart anchor to a first portion of the heart, and deploying a second heart anchor to a second portion of the heart. The first portion of the heart may comprise a papillary muscle 120 as shown in FIG. 2 for example, or may comprise a wall of the patient's heart in examples. The second portion of the heart may comprise a papillary muscle 121 as shown in FIG. 2 for example, or may comprise a wall of the patient's heart in examples. The heart anchors may be deployed in a desired manner.

The tension member may be tensioned to a desired amount between the first heart anchor and the second heart anchor. The tensioning may be set to provide a desired therapeutic effect and the tension member may be locked to the first heart anchor and the second heart anchor at such tension.

In examples, the lengths 514, 516 of the tension member marked in FIG. 3 may be set as desired. For example, a clinician may determine the defined limit that the tension member may expand or stretch to. A length of the tension member may be set to provide a desired therapeutic effect. The length may be preset prior to entry of the patient's heart, or may be adjusted during the procedure to provide a desired therapeutic effect. For example, a clinician may monitor a desired size of the ventricle during a systolic phase and a diastolic phase and may set the lengths 514, 516 based on a desired amount of expansion of the ventricle. In examples, a stop may be set to define a length that the tension member may expand to. In examples, a length of the tension member or material properties of the tension member may be selected to define a length that the tension member may expand to. Other methods to set the lengths 514, 516 may be utilized as desired.

The tension member may be tensioned between the heart anchors to approximate the papillary muscles. A resulting configuration is shown in FIG. 2 for example.

The tension members disclosed herein may be utilized separately, or in combination with other features disclosed herein. In examples, the features of the tension members may be varied, for example, to include coverings on any of the tension members that may reduce the possibility of contact with native chordae or other features of the patient's heart. Other variations may be provided as desired.

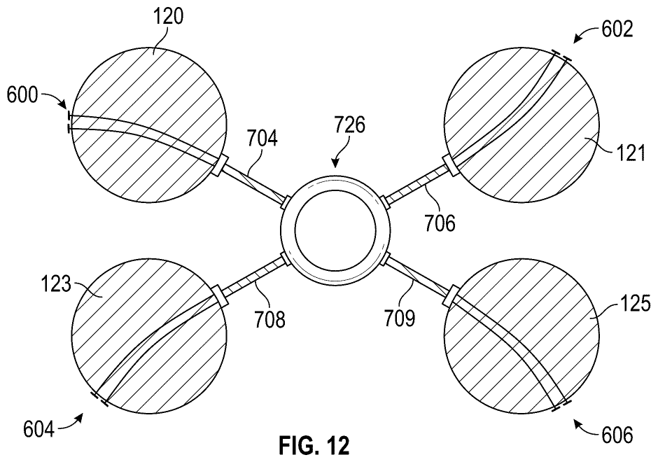

In examples, a tension member may be configured to couple to three or more heart anchors. FIG. 10, for example, illustrates an example of a tension member 700 configured to couple to at least three heart anchors. The tension member 700 may be configured similarly as tension members disclosed herein, and may be configured to expand to accommodate expansion of the ventricle. In examples, the tension member 700 may be configured to expand to a defined limit to accommodate expansion of the ventricle. The tension member 700 may be configured to extend within the ventricle and couple to at least three heart anchors and apply a tension to approximate the papillary muscles to which the tension member 700 is coupled.

The heart anchors may be configured similarly as heart anchors disclosed herein, and may comprise a first heart anchor 600, a second heart anchor 602, and a third heart anchor 604 for example. The first heart anchor 600 may couple to a first portion of the heart, the second heart anchor 602 may couple to a second portion of the heart, and the third heart anchor 604 may couple to a third portion of the heart. The first portion of the heart may comprise a first papillary muscle 120, the second portion of the heart may comprise a second papillary muscle 121, and the third portion of the heart may comprise a third papillary muscle 123. The heart anchors may have any configuration as disclosed herein, including sutures or cuffs or another configuration as desired. In examples, the number of papillary muscles that the tension member may anchor to may be greater as desired, based on a desired therapeutic result. For example, FIG. 12 illustrates an example in which a tension member couples to four heart anchors, each coupled to a papillary muscle. In examples, the heart anchors may be configured to be positioned on other portions of the patient's heart such as a heart wall or other portion of the patient's heart.

The tension member 700 may include an expandable body in the form of a ring 702 that may be coupled to a plurality of tethers 704, 706, 708. The plurality of tethers 704, 706, 708 may extend radially outward from the ring 702 to the respective heart anchors 600, 602, 604. For example, a first tether 704 may be configured to extend radially outward from the ring 702 to the first heart anchor 600, a second tether 706 may be configured to extend radially outward from the ring 702 to the second heart anchor 602, and a third tether 708 may be configured to extend radially outward from the ring 702 to the third heart anchor 604. In examples, the tethers 704, 706, 708 may each be non-expandable, or in examples may be expandable.

The ring 702 may comprise a compliant body that is configured to expand radially outward upon an expansion force being applied by the ventricle 102. The expansion may expand the ring 702 radially outward and may allow for expansion of the ring 702 in the portions 710, 712, 714 between the couplings to the tethers 704, 706, 708. The ring 702 may accordingly allow the papillary muscles 120, 121, 123 to move due to the cardiac cycle.

FIG. 11 illustrates a cross sectional view of the ring 702 in the expanded configuration, allowing the papillary muscles 120, 121, 123 to move due to the cardiac cycle. The ring 702 in examples may comprise an elastic body, which may be configured to provide an elastic force radially inward following expansion of the ring 702. The tension member 700 accordingly may be configured to expand between a first length at a systolic phase of the ventricle and a second length at a diastolic phase of the ventricle, and apply an elastic force between the first length and the second length. For example, as shown in FIG. 11, an elastic ring 716 may be positioned within a cover 718 of the ring 702. The elastic ring 716 may comprise an expandable ring that provides the elastic force radially inward following expansion of the ring 702. The cover 718 may cover the elastic ring 716 and may include couplers 720, 722, 724 that couple to the respective tension member or tether 704, 706, 708.

In examples, the ring 702 may be configured to expand radially outward to a defined limit. For example, the elastic ring 716 may be configured to expand and then expand no further to define the limit that the ring 702 may expand to. In examples, the ring 702 may have other configurations. For example, the ring 702 may include an example as shown in FIGS. 4A-4B or 5A-5B, yet formed into a ring shape. The examples of FIGS. 4A-5B may allow for expansion to a defined limit, and may provide an elastic force that is radially inward.

The features disclosed in regard to FIGS. 4A-5B may be utilized in the examples of FIGS. 10-12. For example, at least a portion of the tension member may include corrugations configured to allow the tension member to expand. The tension member may include a hose having the corrugations. At least a portion of the tension member may include a woven body. The woven body may include a plurality of strips configured to slide with respect to each other to allow the tension member to expand. The plurality of strips may be configured to slide longitudinally to allow the tension member to expand, which may be to a defined limit. In examples, the configuration of the ring may be varied as desired.

FIG. 12 illustrates an example in which a ring 726, configured similarly as the ring 702, is configured to couple to four heart anchors 600, 602, 604, 606 and four respective tethers 704, 706, 708, 709. The heart anchor 606 may couple to a portion of a heart, which may comprise a papillary muscle 125. In examples, a tension member may be utilized that is configured to couple to a greater number of heart anchors and papillary muscles as desired.

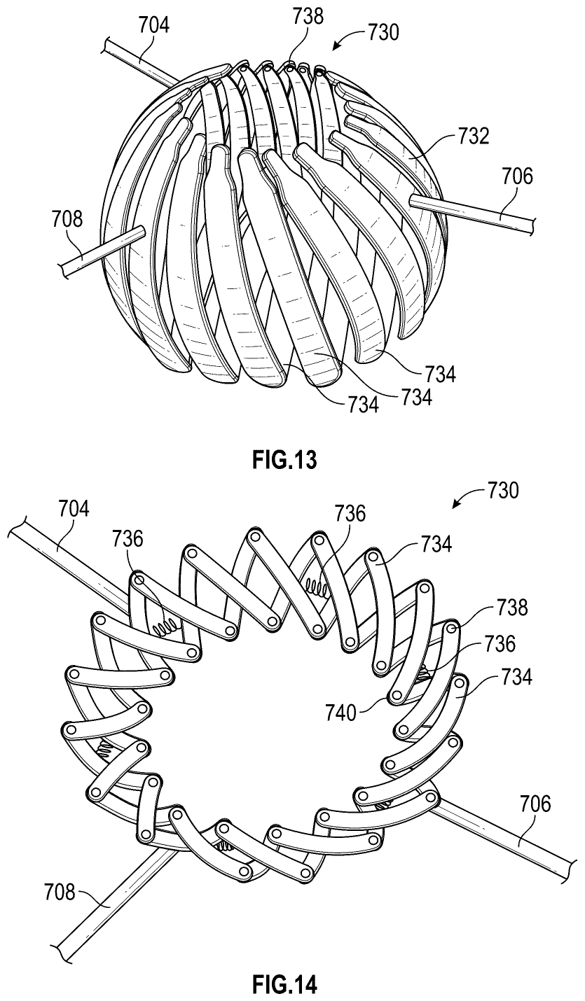

FIG. 13 illustrates an example of a tension member 730 including an expandable body in the form of a ring 732, formed of a plurality of arms 734 that are configured to scissor to allow the ring 732 to expand. The arms 734 may be pivotally coupled to each other and may overlap each other. For example, the angle between the plurality of arms 734 may vary to allow the ring 732 to both increase and decrease in size. The arms 734 may cross each other and be coupled to each other at pivots that may allow the angle between the plurality of arms 734 to vary. In examples, the arms 734 may be configured in a circularized configuration of the arms 552 shown in FIGS. 6A and 6B for example.

The arms 734 may be configured to move from an unexpanded configuration shown in FIG. 13 to an expanded configuration as shown in FIG. 14. The arms 734 may expand radially outward in the expanded configuration.

A portion of the arms 734 may couple to respective tethers 704, 706, 708 that may couple to respective heart anchors 600, 602, 604, as shown in FIG. 10 for example. The tethers 704, 706, 708 may be expandable or non-expandable in examples as desired. The arms 734 may be configured to expand to allow for ventricular expansion.

In examples, the arms 734 may be configured to apply an elastic force radially inward. Such force may be provided as a property of the materials of the arms 734, or one or more springs 736 may be positioned between the arms 734 to provide an elastic force to the plurality of arms 734. FIG. 14, for example, illustrates springs 736 that may be positioned between arms 734 to allow for an elastic force radially inward. The tension member 730 accordingly may be configured to expand between a first length at a systolic phase of the ventricle and a second length at a diastolic phase of the ventricle, and apply an elastic force between the first length and the second length.

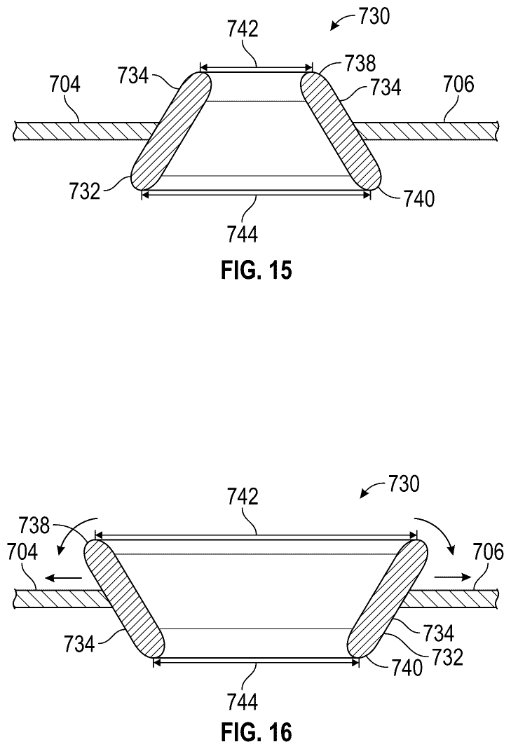

The ring 732 may have an upper end 738 and a lower end 740, and the expansion and contraction of the tension member 730 may vary a distance between the upper end 738 to a different amount than the lower end 740. FIG. 15, for example, illustrates a cross sectional view of an unexpanded configuration of the ring 732 and FIG. 16 illustrates a cross sectional view of the expanded configuration of the ring 732. The ring 732 may expand such that the distance 742 of the upper end 738 of the arms 734 increases to a greater amount than the distance 744 of the lower end 740 of the arms 734. The resulting expansion, however, may allow the tethers 704, 706, 708 coupled to the ring 732 to move radially outward upon the expansion of the ring 732. The arms 734 for example, may rotate outward as represented by the arrows shown in FIG. 16.

The arms 734 may rotate outward to a defined limit. The ring 732 accordingly may expand radially outward to a defined limit. The defined limit may be provided by a stop in examples, similar to a stop 556 as shown in FIGS. 6A and 6B. The stop may be adjustable to adjust the defined limit in examples.

In examples, the ring 732 may be configured to couple to a greater number of tethers and heart anchors, such as four or more tension members and respective heart anchors.

In examples, the heart anchors may be deployed to three or more portions of the patient's heart, which may include three or more papillary muscles or other portions of the heart such as heart walls. A method may include deploying a third heart anchor to a third portion of the heart, and wherein the tension member is configured to couple to the third heart anchor. The third portion of the heart may be a third papillary muscle. A greater number of heart anchors may be deployed as desired, and the tension member may couple to such heart anchors. In deployment, the heart anchors may be delivered to the patient's heart in a minimally invasive manner or another manner as disclosed herein. The heart anchors may be deployed in a desired manner.

Upon deployment of the heart anchors, the tethers shown in FIGS. 10-16 may be tensioned to approximate the papillary muscles. The tension members shown in FIGS. 10-16 may be positioned centrally upon deployment, at a central position between the papillary muscles. The papillary muscles may be positioned to surround the tension member. Other configurations may be utilized as desired. The tensioning may be set to provide a desired therapeutic effect and the tension member may be locked to the heart anchors at such tension.

In examples, a clinician may determine the defined limit that the tension member may expand to. The expansion of the tension member may be preset prior to entry of the patient's heart, or may be adjusted during the procedure to provide a desired therapeutic effect. For example, a clinician may monitor a desired size of the ventricle during a systolic phase and a diastolic phase and may set the expansion of the tension member based on a desired amount of expansion of the ventricle. In examples, a stop may be set to define a length that the tension member may expand to. In examples, a size of the tension member or material properties of the tension member may be selected to define a length that the tension member may expand to. Other methods to set the amount of expansion may be utilized as desired.

The examples of FIGS. 10-16 may be configured to couple to a number of tethers that varies from a number of heart anchors. For example, in one example, three tethers may be utilized that are configured to couple to four heart anchors. A single tether for example, may couple to two heart anchors in such an example. Tethers may be utilized to couple to a greater or lesser number of heart anchors in examples.

In examples, the configuration of the tension members may vary from the configuration shown in FIGS. 10-16. For example, a form of tension member other than a ring may be utilized. A central disc or other body central to the papillary muscles may be utilized that is configured to accommodate expansion of the ventricle. Various other configurations may be utilized as desired.

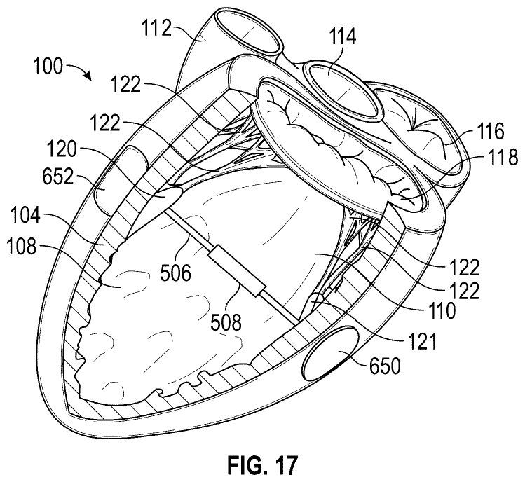

The heart anchors may be coupled to one or more heart walls of the ventricle to approximate the papillary muscles in examples herein. FIG. 17, for example, illustrates the tension member 506 extending between walls of the ventricle to approximate the papillary muscles 120, 121. A first heart anchor in the form of a pad 650 may be positioned on a wall and a second heart anchor in the form of a pad 652 may be positioned on another wall. One or more of the heart anchors may comprise pads configured to be positioned on a wall of the ventricle. The respective walls may comprise posterior and anterior walls of the heart in examples and may be adjacent to papillary muscles to be approximated. Other walls may include an interventricular septum or other walls of the ventricle if desired to approximate the papillary muscles.

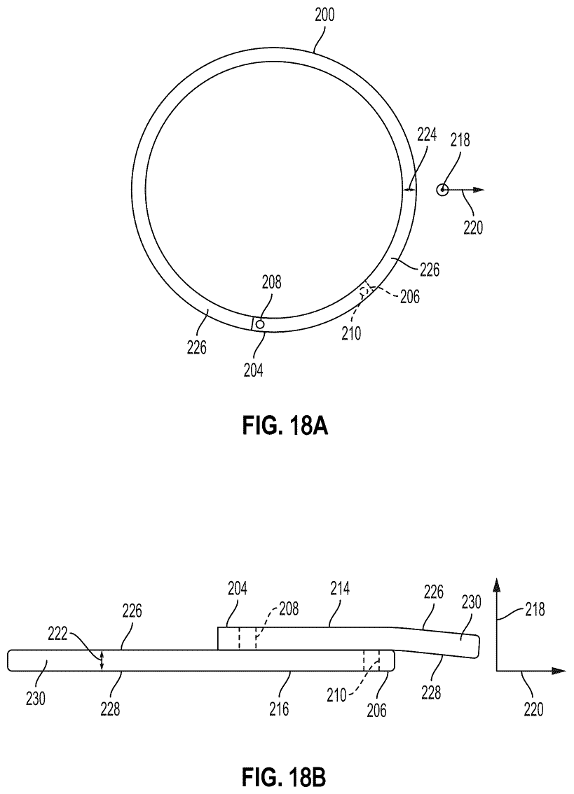

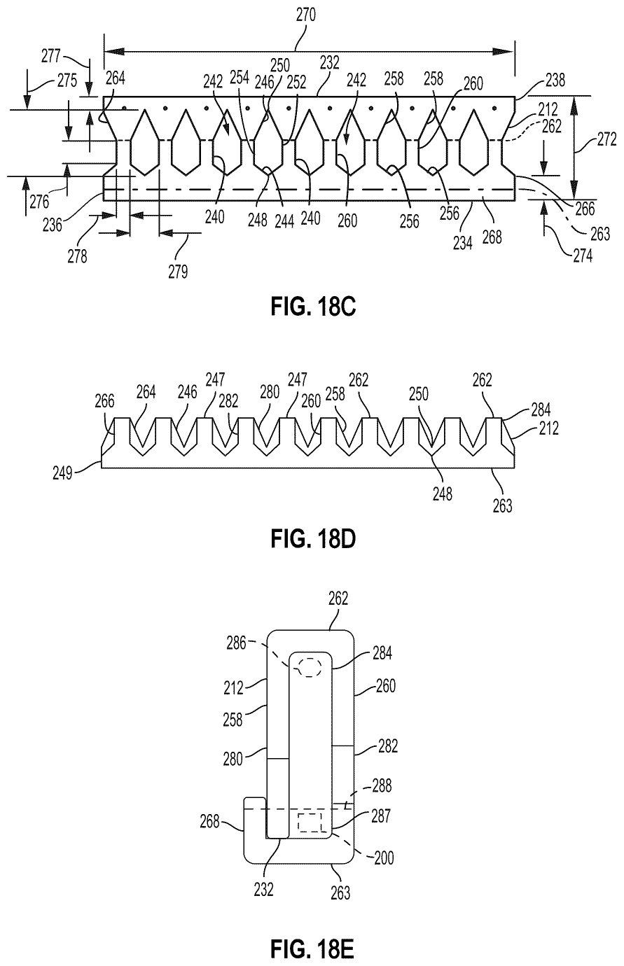

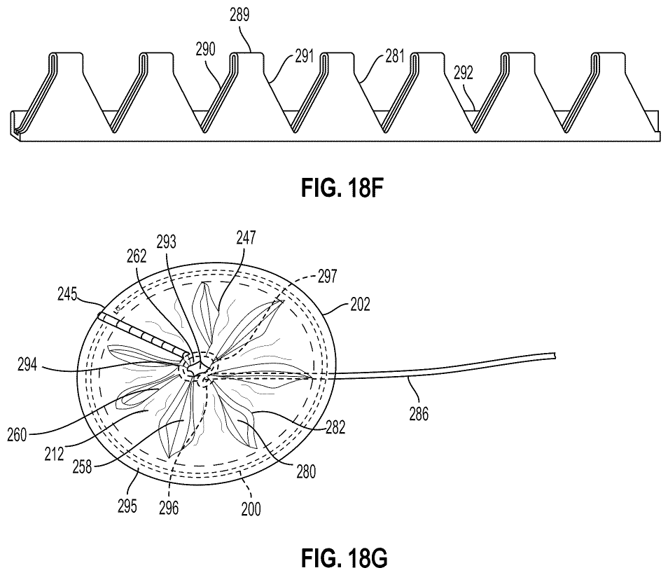

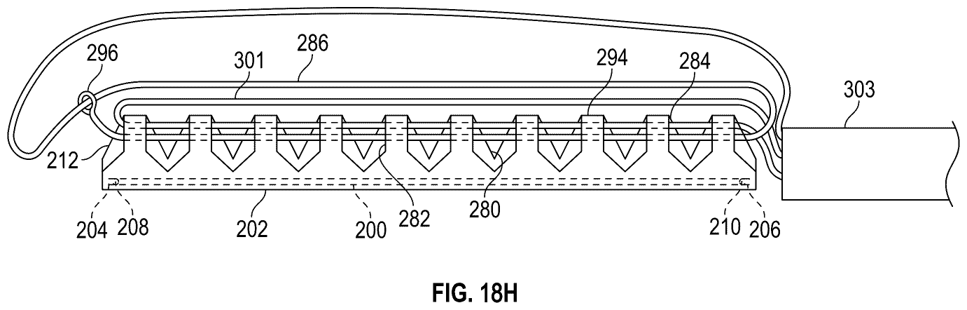

Various forms of heart anchors may be utilized to couple to heart walls in examples. FIGS. 18A-20B, for example, illustrate exemplary features of heart anchors in the form of pads that may be utilized according to examples herein (e.g., as one or more of pads 650, 652 shown in FIG. 17). Various other forms of heart anchors may be utilized as desired. Heart anchors that are configured to couple to heart walls of the ventricle may be utilized in any example disclosed herein, including the examples of FIGS. 1-16. For example, in an example such as shown in FIGS. 10-16, more than two heart anchors may be utilized on a heart wall. Further, in examples, combinations of one or more heart anchor that couple to a heart wall and one or more heart anchors that couple to a papillary muscle may be utilized.

FIG. 18A illustrates a component of a heart anchor, in the form of a pad, that may be utilized in examples herein. Features of heart anchors may be disclosed in U.S. patent application Ser. No. 16/549,957, titled METHODS AND DEVICES FOR VENTRICULAR RESHAPING AND HEART VALVE RESHAPING, filed on Aug. 23, 2019 and published as U.S. Publication No. 2020/0069426, the entire contents of which are incorporated herein for all purposes. FIG. 18A illustrates an example of a ring 200 that may be used in a heart anchor, as may be used in the systems and methods disclosed herein. A heart anchor 202 as may be used in the systems and methods disclosed herein is illustrated in FIG. 18G.

The ring 200 may include a body having a first end 204 and a second end 206 (shown in FIG. 18A in dashed lines, and shown in FIG. 18B). The ring 200 may be configured to move from a linearized configuration (as shown in FIG. 18H) to a ring-shaped configuration, as shown in FIG. 18A. The first end 204 may include an opening 208 extending through the ring 200 at or proximal the first end 204 and an opening 210 (shown in FIGS. 18A and 18B in dashed lines) extending through the ring 200 at or proximal the second end 206. The openings 208, 210 may comprise couplers for coupling to the cover 212 (shown in FIGS. 18G and 18H).

FIG. 18B illustrates a side view of the ring 200 in the ring-shaped configuration shown in FIG. 18A. Portions 214, 216 of the ring 200 may overlap when the ring 200 is in the ring-shaped configuration. The portions 214, 216 may overlap in the axial dimension 218, as opposed to the radial dimension 220. The portions 214, 216 may overlap such that the portions 214, 216 that overlap include the ends 204, 206. From a top view (as shown in FIG. 18A), the portions may overlap such that the edges of the body of the ring 200 have a matching profile as viewed from the top. The edges of the body of the ring 200 may be aligned with each other in the radial dimension 220. The edges of the body of the ring are not offset from each other at the overlapping portions in the radial dimension 220. Thus, the ring 200 may appear as a continuous ring, which may have a circular shape or other shape as desired. Some examples of the ring include any suitable closed shape, which can be generally flat, as illustrated in FIGS. 18A and 18B, or can have a three-dimensional shape, for example, that accommodates one or more anatomical features.

In some examples, a first end portion of the ring can overlap a second end portion of the ring where at least one of the first end portion or the second portion is adjacent to or spaced from the respective end. For example, in some rings, at least one edge of the first end portion is offset or at an angle to at least one edge of the second end portion at the overlap. In other examples, the overlapping portions can include both ends of the first and second end portions where at least one edge of the first end portion is offset from an edge of the second end portion.

The overlapping portions 214, 216 may contact each other, and one of the overlapping portions may provide a support against force for the other overlapping portion. For example, a force applied to portion 214 may be resisted by portion 216 at the overlap, and a force applied to portion 216 may be resisted by portion 214 at the overlap. The overlapping portions 214, 216 may provide support for the ring 200 upon a force being applied in the axial dimension.

The overlapping portions 214, 216 may overlap to a desired amount. In one example, the overlapping portions 214, 216 may overlap to at least about 5 degrees of the ring 200. In one example, the overlapping portions 214, 216 may overlap to at least about 10 degrees, to at least about 20 degrees, to at least about 40 degrees, or to at least about 60 degrees of the ring 200, or to a different amount as desired. In one example, the entirety of the ring 200 may overlap such that the overlapping portions 214, 216 comprise the entirety of the ring, for example, about 360 degrees, or even greater than 360 degrees. In one example, the ring 200 may be configured to have a single overlap, as shown in FIG. 18A, which may reduce the amount of material comprising the ring 200 and may ease the transition between the linearized configuration and the ring-shaped configuration. As will be apparent in the discussion below, the degree of overlap can change when the ring is in use. For example, tensioning and/or applying a load to the cover can reduce a diameter/circumference of the ring, thereby increasing the overlap in some examples.

The ring 200 may have a thickness 222 (in the axial dimension 218) and may have a width 224 (in the radial dimension 220) (as marked in FIG. 18A). The thickness 222 may be between about 0.2 and about 0.4 millimeters, although in other examples other thicknesses 222 may be utilized. In one example, the thickness 222 may be about 0.3 millimeters. In some examples, the thickness can be non-uniform along a length/circumference of the ring. For example, in some rings, at least one of the overlapping portions can be thinner than a non-overlapping portion of the ring. The width 224 may be between about 0.3 and about 0.5 millimeters, although in other examples other widths 224 may be utilized. In one example, the width 224 may be about 0.4 millimeters. In some examples, the width is non-uniform along a length/circumference of the ring, for example, wider at at least one of the overlapping portions. The ring 200 may be sized as desired, and may be configured to be a relatively thin ring that is flexible to allow for ease of movement of the ring 200.

Referring to FIG. 18B, the ring 200 may have a flattened shape with a substantially planar top surface 226 and a substantially planar bottom surface 228 facing opposite the top surface 226. The ring 200 at the overlapping portions 214, 216 may have the top surface 226 of portion 216 face towards the bottom surface 228 of portion 214. The terms “top” and “bottom” may be used interchangeably. Side surfaces 230 may connect the top surface 226 to the bottom surface 228. The body of the ring 200 may have a rectangular profile when viewed in cross section.

The ring 200 may be made of a material that is flexible, such that the ring may move from the linearized configuration (as shown in FIG. 18H) to the ring-shaped configuration (as shown in FIG. 18A). The ring 200 may be made of an elastic material to move from one configuration to the other relaxed or default configuration. In one example, the ring 200 may be made of a super elastic or shape-memory material, which may include a shape-memory alloy, to allow the ring to move from the linearized configuration to the ring-shaped configuration. The shape-memory material may be a material such as nitinol or another shape-memory material. The ring 200 may be configured to automatically move from the linearized configuration to the ring-shaped configuration, as the shape-memory material may automatically move to the shape-set ring-shaped configuration.

A linearized configuration is shown in FIG. 18H. In this configuration, the portions 214, 216 of the ring 200 may be separated from each other and do not overlap. The ends 204, 206 of the ring 200 do not overlap. The ring 200 in FIG. 18H may be in one form of linearized configuration, however, other forms may be utilized. For example, two opposite portions of the ring 200 in the ring-shaped configuration may be squeezed together towards a center portion of the ring such that the opposite portions of the ring-shaped body come together at the center portion and the ring 200 is linearized. Other forms of linearization may be utilized. The ring 200 in the linearized configuration may not be entirely straightened, although in other examples, the ring 200 may be entirely straightened (as shown in FIG. 18H). As such, the term “linearized” refers to any configuration that is suitable for delivery and deployment through a catheter or other minimally invasive or percutaneous delivery system, and does not require that the ring or any portion thereof is substantially straight or linear. In some examples, no portion of the ring is substantially straight or linear in the linearized configuration. For example, all or a portion of a ring can adopt a helical, sinusoidal, and/or other curved shape in the linearized configuration. Consequently, the terms “delivery configuration” and “open configuration” can also be used to describe some examples. The ring 200 in the linearized configuration may be configured to fit within the lumen of a deployment apparatus 303, as marked in FIG. 18H.

FIG. 18C illustrates a top view of the cover 212 unfolded. The cover 212 may include a top edge 232 and an opposite bottom edge 234. Side edges 236, 238 may extend from the top edge 232 to the bottom edge 234. The terms “top” and “bottom” may be used interchangeably.

The cover 212 may include a plurality of cut-outs 240 defining openings 242 in the cover 212. The cut-outs 240 may be in the form of a pattern of shapes. The shapes, as shown in FIG. 18C, may be asymmetric diamonds. The asymmetric diamonds may include opposite triangular portions 244, 246. The triangular portions 244 may have an angle and side lengths from the central vertex 248 that is different than the angle and side lengths from the central vertex 250 of the triangular portion 246. The angle from the central vertex 248 is greater than the angle from the central vertex 250, and the side lengths from the central vertex 248 are shorter than the side lengths from the central vertex 250. The side lengths of the triangular portions 244, 246 extend to straight side portions 252, 254.

The cut-outs 240 may leave the remaining portion of the cover 212 with trapezoidal portions 256, 258 having differing heights and side lengths. The height and side lengths of the trapezoidal portions 256 may be less than the height and side lengths of the trapezoidal portions 258. The trapezoidal portions 256, 258 may be connected with rectangular portions 260.

The pattern of cut-outs 240 may be repeated along the length of the cover 212. The shape and pattern of cut-outs 240 and shape and pattern of the remaining portions of the cover 212 may be varied from the shapes and pattern shown in FIG. 18C as desired. In examples in which the ring has a non-circular ring-shaped or closed configuration, the particular details of the cover can differ, for example, the patterns of shapes defined by the cut-outs and/or their dimensions.

The cover 212 may include a fold portion 262 marked in dashed lines in FIG. 18C. The cover 212 may be configured to fold at the fold portion 262. The portion of the cover 212 shown above the fold portion 262 may comprise an overlapping portion 264 and the portion of the cover 212 shown below the fold portion 262 and above the dot-dashed line may comprise an overlapping portion 266. The overlapping portion 266 may overlap the overlapping portion 264 when the cover 212 is folded at the fold portion 262. The cover 212 may include a fold portion 263 marked in dot-dashed lines in FIG. 18C. The cover 212 may include an overlapping portion 268 indicated below the dot-dashed line in FIG. 18C. The overlapping portion 268 may be configured to overlap the top edge 232 of the cover 212 and a portion of overlapping portion 264 when the cover 212 is folded upon the fold portion 262.

The dimensions of the cover 212 may be set as desired. The dimensions may include a length 270. The length 270 may extend from the side edge 236 to the side edge 238. The length 270 may be between about 100 millimeters and about 70 millimeters, and in other examples, may have a greater or lesser size as desired. In one example, the length 270 may be about 88 millimeters. The dimensions may include a width 272 of the unfolded cover 212. The width 272 may extend from the top edge 232 to the bottom edge 234. The width 272 may be between about 20 millimeters and about 30 millimeters, and in other examples, may have a greater or lesser size as desired. In one example, the width 272 may be about 22 millimeters.

The dimensions may include a width 274 of the cover 212 from the bottom edge 234 to the lower end of the cut-outs 240. The width 274 may be between about 4 millimeters and about 7 millimeters, and in other examples, may have a greater or lesser size as desired. In one example, the width 274 may be about 5.5 millimeters. The dimensions may include a width 275 of the cut-outs 240. The width 275 may be between 10 about millimeters and about 15 millimeters, and in other examples, may have a greater or lesser size as desired. In one example, the width 275 may be about 13.5 millimeters. The dimensions may include a width 276 of the rectangular portions 260. The width 276 may be between about 3 millimeters and about 7 millimeters, and in other examples, may have a greater or lesser size as desired. In one example, the width 276 may be about 5 millimeters. The dimensions may include a width 277 of the cover 212 from the top edge 232 to the upper end of the cut-outs 240. The width 277 may be between about 1 millimeter and about 5 millimeters, and in other examples, may have a greater or lesser size as desired. In one example, the width 277 may be about 3 millimeters.

The dimensions may include a thickness 278 of the rectangular portions 260. The thickness 278 may be between about 1 millimeter and about 5 millimeters, and in other examples, may have a greater or lesser size as desired. In one example, the thickness 278 may be about 3 millimeters. The dimensions may include a thickness 279 of the cut-outs 240. The thickness 279 may be between about 3 millimeters and about 8 millimeters, and in other examples, may have a greater or lesser size as desired. In one example, the thickness 279 may be about 6 millimeters.

FIG. 18D illustrates the cover 212 having been folded at the fold portion 262. The overlapping portion 266 overlaps the overlapping portion 264 (and the overlapping portion 264 overlaps the overlapping portion 266). The overlapping portions 264, 266 form respective layers, including a first layer 280 and a second layer 282 that overlap and may be in contact with each other. The cover 212 may be folded such that the central vertices 250 may be brought towards the central vertices 248, and the trapezoidal portions 258 overlap the rectangular portions 260. The triangular portions 246 of the cut-outs may form triangular-shaped gaps between the trapezoidal portions 258. The cover 212 in this configuration includes a plurality of protrusions 247 extending from a connecting portion 249 of the cover 212.

The cover 212 at the fold portion 262 may form a coupler 284 for coupling the cover 212 to a tension member 286 (as shown in FIG. 18G). The cover 212 at the fold portion 263 may form a coupler 287 (marked in FIG. 18E) for coupling to the ring 200. The couplers 284, 287 may comprise folded material at the fold portions 262, 263 that the respective tension member 286 and ring 200 may be passed through.

FIG. 18E illustrates a side view of the cover 212 in the configuration shown in FIG. 18D. The position of the tension member 286 (if coupled to the cover 212) is shown in dashed lines at the top of the cover 212 and the position of the ring 200 (if coupled to the cover 212) is shown in dashed lines at the bottom of the cover. The overlapping layers 280, 282 are visible. The overlapping portion 268 overlaps the edge 232 of the cover 212. The overlap of the overlapping portion 268 forms the coupler 287 in the form of a loop at a bottom end of the cover 212. The bottom end, when the ring 200 is in the ring-shaped configuration, may comprise a peripheral portion of the cover 212. Connectors 288 may extend through the layers 280, 282 and overlapping portion 268 to secure the loop in position at the bottom end of the cover 212. The connectors 288 may comprise sutures or other form of stitching, or another form of connector that connects the overlapping layers 280, 282 and overlapping portion 268. The connectors 288 may pass through the openings 208, 210 in the ring 200 to securely connect the ring 200 to the cover 212. The ring 200 may be positioned between the connectors 288 and the fold portion 263, and sandwiched between the layers 280, 282.

The cover 212 at the fold portion 262 forms a coupler 284 in the form of a loop at the top end of the cover 212. The top end, when the ring 200 is in the ring-shaped configuration, may comprise a central portion of the cover 212. The tension member 286 may pass through the coupler 284 and may be sandwiched between the layers 280, 282.

FIG. 18F illustrates an example of a cover 281 having a different configuration of cut-outs than shown in the example of FIGS. 18C-18E, 18G and 18H. The cut-outs in the example of FIG. 18F are symmetrical as folded upon the fold portion 289. The remaining portions of the cover 281 include a first layer 290 and a second layer 291 that have the same symmetrical shapes of trapezoidal portions coupled to rectangular portions (that include the fold portion 289). An overlapping portion 292 may overlap the layers 290, 291 and may form a coupler for coupling to the ring 200, in a similar manner as the example of FIGS. 18C-18E, 18G and 18H. The fold portion 289 may form a coupler for coupling to the tension member 286, in a similar manner as the example of FIGS. 18C-18E, 18G and 18H. The configuration of cover 281 may be utilized with the systems and methods disclosed herein, in a similar manner as cover 212. The shape and configuration of the covers 212, 281 shown in FIGS. 18C-18H may be varied as desired.

The covers 212, 281 may be flexible and configured to move with the ring 200 as it moves from the linearized configuration to the ring-shaped configuration. The covers 212, 281 may be made of a flexible material, which may include, for example, a cloth or fabric. The flexible material may be woven or non-woven. The flexible material may include materials such as ultra-high-molecular-weight polyethylene (UHMwPE) (for example, DYNEEMA® fabric or laminate, Koninklijke DSM, the Netherlands) or polyethylene terephthalate (PET, for example, DACRON® fabric, Invista, Wilmington, Delaware). In other examples, other flexible materials may be utilized.

FIG. 18G illustrates the ring 200 in a ring-shaped configuration, with the ring 200 coupled to the cover 212, and the tension member 286 coupled to the cover 212. Portions of the ring 200 coupled to the cover 212 may overlap, in a manner discussed previously. Portion 245 comprises an overlapping portion of the ring 200 and the cover 212. The cover 212 extends inward from the ring 200 in the ring-shaped configuration.

The tension member 286 is coupled to the cover 212 at the fold portions 262. The tension member 286 is drawn away from the cover 212 such that the cover 212 is drawn towards a central opening 293 of the cover 212. The tension member 286 accordingly may cinch the cover 212 towards the central opening 293. In some examples, the cover 212 can in turn pull on the ring 200, reducing a diameter/circumference thereof. The cover 212 is in a disc-shaped configuration. The cover 212 in this configuration includes a central portion 294 and a peripheral portion 295. The overlapping layers of material of the cover 212 (the layers 280, 282) extend from the peripheral portion 295 to the central portion 294. The fold portions 262 are positioned at the central portion 294, and the ring 200 is positioned in the peripheral portion 295.

The trapezoidal portions 258 of the layer 280 may be placed adjacent each other such that the gaps between the trapezoidal portions 258 shown in FIG. 18D are closed. The cover 212 accordingly may comprise a closed surface extending from the peripheral portion 295 to the central portion 294. The protrusions 247 are adjacent each other and extend from the peripheral portion 295 to the central portion 294.

The tension member 286 may comprise a portion of a heart splint, and may be configured to provide tension between the anchors of a heart splint. The tension member 286 may comprise a tether, and may be in the form of a cord, or other form of tension member. The tension member 286 may comprise a portion of the tension member 506 shown and described herein for example.

The tension member 286 may be made of a flexible material, which may include ultra-high-molecular-weight polyethylene (UHMwPE) (for example, FORCE FIBER® suture, Teleflex, Wayne, Pennsylvania or DYNEEMA® fiber, Koninklijke DSM, the Netherlands), among other flexible materials. The tension member 286 may include a body, and may include a coupling device 296 at its end that may couple the tension member 286 to itself. The coupling device 296 may comprise a loop that the body of the tension member 286 passes through, such that as the body of the tension member 286 is pulled, a size of a loop 297 formed by the tension member 286 being threaded through the fold portions 289 of the cover 212 reduces in size. The portion of the tension member 286 forming the loop 297 passes through the coupler 284 (marked in FIG. 18D) positioned at the central portion 294 of the cover 212. Thus, as the tension member 286 is pulled, the size of the loop 297 reduces, and accordingly the cover 212 is cinched and pulled radially towards the central opening 293 of the cover 212. The anchor 202 in the configuration shown in FIG. 18G may have a diameter of between about 20 millimeters and about 25 millimeters, although other diameters may be utilized as desired. In one example, the anchor 202 may have a diameter of about 22 millimeters.

The cover 212 may be configured to be drawn towards the central opening 293 such that the central opening 293 entirely closes. The tension member 286 may extend from the cover 212 at the central portion 294 of the cover 212.

FIG. 18H illustrates the ring 200 in a linearized configuration. The ring 200 is extended such that the ends 204, 206 are separated from each other. The tension member 286 is visible extending through the coupler 284 of the central portion 294. The anchor 202 is in a linearized configuration.

A deployment member 301 may be utilized to deploy the cover 212 of the anchor 202. The deployment member 301 may comprise a tether, and may be in the form of a cord, or other form of deployment member. The deployment member 301 may be looped, and may be coupled to the cover 212 at the coupler 284. The deployment member 301 may be looped through the coupler 284 in a manner shown in FIG. 18H. The deployment member 301 may be pulled to cinch or draw the cover 212 towards the central opening 293 of the cover 212 in a similar manner as discussed above regarding the tension member 286. The anchor 202, upon being positioned in the lumen of a deployment apparatus, may have the cover 212 flattened. The deployment member 301 may close or otherwise cinch the cover 212. The anchor 202 may be held against the end of the deployment apparatus 303 when the deployment member 301 is pulled, to support the anchor 202 in position. The ring 200, however, may also be configured to automatically move to or towards the ring-shaped configuration.

The anchor 202 may accordingly be configured to move from an unexpanded configuration to an expanded configuration. The unexpanded configuration may comprise the configuration in which the ring 200 is in the linearized configuration, and the anchor 202 is accordingly linearized. The expanded configuration may be the configuration in which the ring 200 is in the ring-shaped configuration and the cover 212 is in a disc-shaped configuration. In other examples, other unexpanded and expanded configurations may be utilized. In an expanded configuration, an anchor may have a larger diameter or other dimensions. In unexpanded configuration, the anchor may have a smaller diameter or other dimensions and may be configured to fit within the lumen of a deployment apparatus. The configurations of anchor may vary from that shown in FIGS. 18G and 18H.

The anchor 202 may beneficially be configured such that the cover 212 bears the majority of the force against the anchor 202 when the tension member 286 is tensioned. The ring 200 may be configured to provide support for the shape of the cover 212, but otherwise may bear a lesser portion of the force against the anchor 202. The overlapping portions of the ring 200 may beneficially provide enhanced strength for the ring 200.

The relatively thin shape of the ring 200 may allow the ring 200 to be flexible to fit within the lumen of a deployment apparatus. The ring 200 may be able to be positioned within the lumen of a deployment apparatus with a relatively low force, and may be positioned within the lumen manually. The ring 200 may be sufficiently flexible to be hand-loaded into a deployment apparatus.

The anchor 202 may have a variety of uses, including use as a portion of a heart splint as may be disclosed herein.

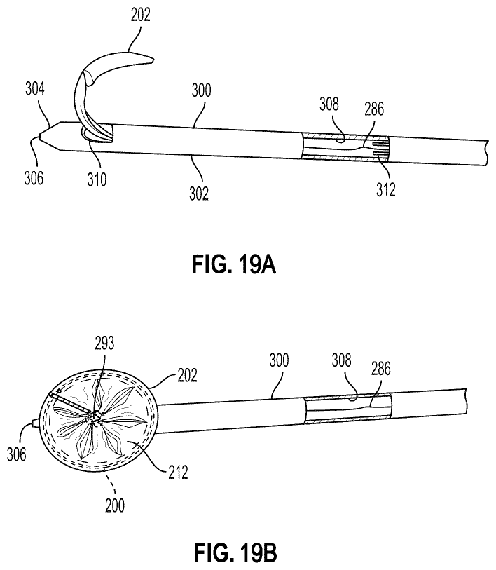

FIG. 19A illustrates an example of a deployment apparatus 300 that may be utilized according to examples herein. The deployment apparatus 300 may comprise a puncturing device include a body portion 302 and a distal end 304. The deployment apparatus 300 may be utilized in a method of applying heart anchors to a patient's heart.

The distal end 304 of the deployment apparatus 300 may include a puncturing tip 306. The puncturing tip according to examples herein may be for puncturing one or more surfaces of a heart, including an external posterior surface and an external anterior surface of the heart. The deployment apparatus 300 may include an interior lumen 308 and may include an opening 310 along the body portion 302 positioned adjacent the puncturing tip 306. The interior lumen 308 may comprise an implant retention area for retaining a heart anchor in an unexpanded configuration. The deployment apparatus 300 may include a push device 312 for passing through the lumen 308 for pushing a heart anchor 202 out of the opening 310.

The body portion 302 may have the shape of an elongate rigid rod. The body portion 302 may be sufficiently rigid to withstand the force of penetrating through a portion of a patient's heart.

The push device 312 may be configured to pass through the lumen 308 with an internal lumen to allow the tension member 286 to pass through the lumen 308 and the internal lumen, and be accessible for tensioning by a user.

The interior lumen 308 may be configured to retain the anchor 202 in the unexpanded or linearized configuration within the lumen 308. The anchor 202 may be positioned within the lumen 308 such that as the anchor 202 is pushed out of the lumen 308 with the push device 312, the tension member 286 remains in the lumen 308 and a portion of the tension member 286 remains accessible to be pulled to move the cover 212 towards the central opening 293 of the cover 212 as discussed herein. The ring 200 (marked in FIG. 18G) of the anchor 202 may be configured to automatically move to the ring-shaped configuration, as discussed herein. In examples, a separate deployment member 301 as shown in FIG. 18H may be utilized to be pulled to move the cover 212 towards the central opening 293 of the cover. However, in examples, the tension member 286 itself may be pulled.

The anchor 202 may be configured to move from the unexpanded configuration to the expanded configuration adjacent the opening 310. In one example, multiple anchors 202 may be positioned within the lumen 308 and may be pushed out in sequence.

FIG. 19B illustrates the anchor 202 passed out of the opening 310 and in the expanded configuration in which the ring 200 (marked in FIG. 18G) of the anchor 202 is in the ring-shaped configuration.

The deployed anchor 202 may be coupled to a tension member including an expandable body or otherwise configured to expand as disclosed herein.

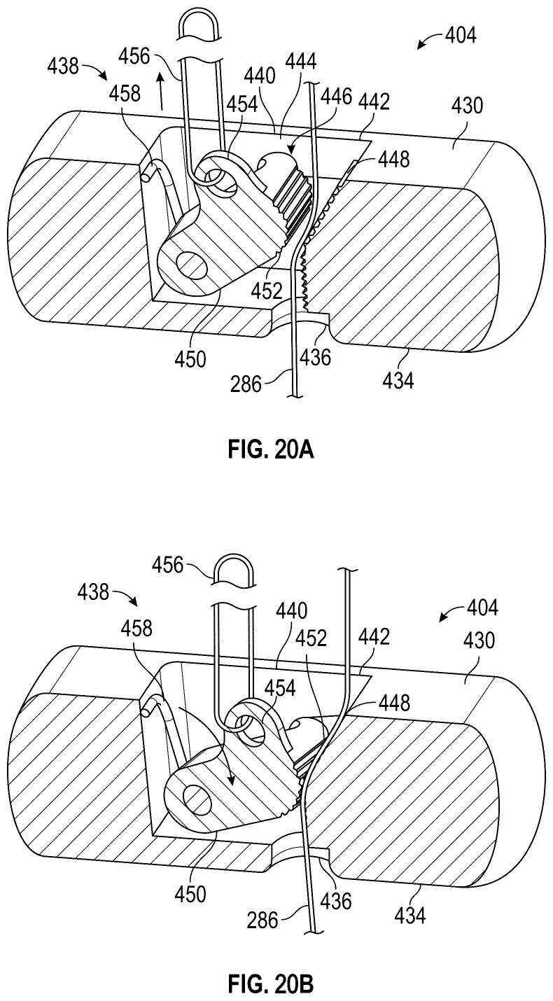

FIG. 20A illustrates a cross sectional view of an example of a heart anchor 404 that may be utilized according to examples herein. The heart anchor has the configuration of a pad, yet retains a mechanical lock 438 therein. A cross sectional view showing the lock 438 and a receiver 440 is provided. The receiver 440 may be configured to receive the proximal portion of a tension member, which may comprise the tension member 286 or another form of tension member disclosed herein.

The receiver 440 may include an opening 442 in the top surface 430 of the heart anchor 404 and may include the opening 436 in the bottom surface 434 of the heart anchor 404. The receiver 440 may include one or more side walls 444 that define a cavity 446 in the heart anchor 404. One of the side walls 444 may include a locking surface 448. The locking surface 448 may comprise a surface for the tension member to be pressed against upon operation of the lock 438. The locking surface 448 may include a grip surface, which may include ridges or another gripping structure that may improve a grip of the locking surface 448.

The lock 438 may be positioned within the receiver 440. The lock 438 may be coupled to the heart anchor 404. The lock 438 may be configured to vary from an unlocked state in which the tension member is unlocked in the receiver 440 to a locked state in which the tension member is locked in the receiver 440. The lock 438 may be configured to move from the locked state to the unlocked state.

The lock 438 may include a rotatable body 450, which may be configured to rotate about a pivot. The pivot may comprise an axle extending through the rotatable body 450, or another form of pivot. The rotatable body 450 may be configured to rotate within the cavity 446 of the receiver 440. The rotatable body 450 may comprise a cam body with a surface of the body 450 comprising a locking surface 452. The cam body may allow the force from the lock 438 against the tension member to increase as tension is increased upon the tension member. The lock 438 may comprise a cam-lock in examples. The locking surface 452 may comprise a surface to press against the tension member, and press the tension member against the locking surface 448, to lock the tension member in position within the receiver 440. The locking surface 452 may include a grip surface, which may include ridges or another gripping structure that may improve a grip of the locking surface 452.

The lock 438 as such may include a ratchet mechanism, that may be configured to allow the tension member to be drawn through the heart anchor 404 in a direction, and resist movement of the tension member in an opposite direction. As such, the tension member may be drawn in a proximal direction to tension the tension member, with the lock 438 resisting the tension member from loosening in the distal direction.

In examples, the lock 438 may include a connector 454 as shown in FIGS. 20A and 20B, for example, for coupling with a lock retainer member 456. The connector 454 may include an aperture for the lock retainer member 456 to be passed through. The lock retainer member 456, for example, may comprise a tether such as a looped cord that couples to the connector 454. The lock retainer member 456 may be tensioned by a user to release the lock 438, and may be released or cut to set the lock 438.

The lock 438 may include a biasing device 458. The biasing device 458 may bias the lock 438 to a locked state, in which the rotatable body 450 is pressed towards the locking surface 448. The rotatable body 450 in the locked state may press the tension member against the locking surface 448 to prevent movement of the tension member and to lock the tension member to the anchor 404. The biasing device 458 may comprise a spring, or other form of biasing device as desired.

The lock retainer member 456 may be pulled to oppose the biasing force of the biasing device 458 and may retain the lock 438 in an unlocked state. The lock retainer member 456 may be configured to couple to the rotatable body 450 to hold the rotatable body 450 in the unlocked state. Such an unlocked state is shown in FIG. 20A. The locking surface 452 of the rotatable body 450 is pulled away from the locking surface 448 of the receiver 440 and the tension member may slide within the receiver 440 and through the openings 436, 442.

The tension member may be slid within the receiver 440 to tension the tension member before the lock 438 is moved to the locked state. Upon a desired amount of tension being reached, the lock retainer member 456 may be moved. The movement of the lock retainer member 456 towards the anchor 404 may allow the biasing device 458 to move the lock 438 to the locked state.

The heart anchor 404 may comprise a pad in which the bottom surface 434 forms a wide contact surface for contact with the external surface of the heart loo, such as an external anterior surface or external posterior surface. The heart anchor 404 may have a disk shape, or may have other shapes as desired. The heart anchor 404 may be configured to have a static size that does not move from an unexpanded configuration to an expanded configuration. In examples, the heart anchor 404 may be configured to move from an unexpanded configuration to an expanded configuration. Variations in the lock 438 and the heart anchor 404 may be provided in examples.

The heart anchor 404 may be utilized with any example of a heart splint disclosed herein, including an example including an expandable body as disclosed herein.

A resulting configuration of a heart splint utilizing heart anchors on a wall of the patient's heart is shown in FIG. 17 for example. The heart anchor 650 shown in FIG. 17, for example may be similar to a heart anchor 202 shown in FIG. 18G. The heart anchor 652 shown in FIG. 17, for example, may be similar to a heart anchor 404 shown in FIGS. 20A and 20B. A tension member extending from the heart anchor 650 to the heart anchor 652 may be tensioned and locked at the heart anchor 652 to approximate the papillary muscles. The heart anchors may be positioned in a variety of locations, including anterior and posterior walls or on an interventricular septum or other walls as desired. The tension member may be configured to expand, as disclosed herein, to allow for expansion of the ventricle. The properties of the tension member, including a length that the tension member may expand to, may be set in a similar manner as discussed with regard to FIGS. 2-16. Various other forms of heart anchors and locations of anchoring may be provided as desired.

The heart splints disclosed herein may be deployed from deployment apparatuses, which may pass into the patient's body in a minimally invasive manner.

Any heart splint disclosed herein may be deployed in a beating heart procedure in examples. A beating heart procedure may allow a user, such as a surgeon, to better determine the effects of the approximation of the papillary muscles, to better determine the possible results of such a procedure. Such a method may comprise an improvement over methods of operating on an arrested heart, in which hemodynamics may not be monitored in real time. The systems, apparatuses, and methods disclosed herein, however, may be utilized on an arrested heart in examples.

In examples, the systems, apparatuses, and methods disclosed herein are disclosed in regard to approximation of the papillary muscles of the left ventricle. However, in examples, approximation of papillary muscles of the right ventricle may be performed in a similar manner. Such approximation may address tricuspid valve regurgitation of the tricuspid valve. The heart anchors may be positioned on an external surfaces, such as an external anterior surface and/or an external posterior surface of the right ventricle in such a manner, in a similar manner as disclosed herein.

The methods disclosed herein may beneficially provide for treating a dilated ventricle of the heart, while providing a minimally invasive procedure. Under the methods disclosed, a full sternotomy may not be required, and entry into the left ventricle may comprise an endovascular entry into the patient's heart. The application of the splint may comprise a beating-heart repair of the left ventricle. The method may include reshaping a ventricle of the heart by applying pressure to the heart to reshape the geometry of heart. Endovascular or transcatheter methods may be utilized. Percutaneous entry of the patient's body may occur. In one example, a full sternotomy may be performed if desired.

The apparatuses and other components disclosed herein may comprise one or more systems. The systems may be utilized in a variety of methods. The methods may include the methods disclosed herein. The methods may include a method for treating ventricular dilation and/or mitral regurgitation and/or tricuspid regurgitation. The methods may include deploying a heart splint.

The steps disclosed herein are illustrative, and may be modified, varied, reordered, or excluded as desired. The “steps” referred to herein may include multiple steps, or may comprise portions of steps.

The heart splints as disclosed herein may be utilized in combination with heart valve prosthetics, heart valve repair implants, or other devices, systems, or apparatuses disclosed herein may be utilized as desired. For example, such heart splints may be utilized in combination with an annuloplasty ring, or other annuloplasty devices, or other form of device for repair of a heart valve annulus.

The “user” as discussed herein may comprise a user of the systems and apparatuses disclosed herein, which may include a surgeon, or another individual such as a medical professional who may operate the systems and apparatuses disclosed herein, without limitation.

The present disclosure offers numerous advantages over existing treatments for various heart conditions, including valve incompetencies. The devices disclosed herein do not require the highly invasive procedures of current surgical techniques. For instance, the treatments described herein do not require removing portions of heart tissue, nor do they necessarily require opening the heart chamber or stopping the heart during operation. The methods of the present disclosure may comprise beating-heart repair of or treatment of the patient's heart. For these reasons, the treatments and techniques for implanting the devices of the present disclosure convey a reduced risk to the patient as compared with other techniques. The less invasive nature of the treatments and techniques and tools of the present disclosure may further allow for earlier intervention in patients with heart failure and/or valve incompetencies. While often discussed herein in terms of mitral valve treatments, the systems, devices, methods, etc. may be used to treat other heart valves, heart conditions, enlargement of other organs, etc.

Although the present disclosure is discussed in connection with treating the mitral valve and tricuspid valve of the heart, the present disclosure may be applied to various chambers of the heart and for other valves of the heart for similar purposes. More broadly, the systems, apparatuses, methods, etc. disclosed herein may be used in other applications to change the geometries and/or stresses of other parts of the body (e.g., a stomach, bladder, or other part of the body).

The apparatuses and other devices disclosed herein may be practiced separately as desired. In addition, the methods herein are not limited to the methods specifically described, and may include methods of utilizing the systems, apparatuses, and devices disclosed herein.

For purposes of this description, certain aspects, advantages, and novel features of the examples of this disclosure are described herein. The disclosed methods, apparatuses, and systems should not be construed as limiting in any way. Instead, the present disclosure is directed toward all novel and nonobvious features and aspects of the various disclosed examples, alone and in various combinations and sub-combinations with one another. The methods, apparatuses, and systems are not limited to any specific aspect or feature or combination thereof, nor do the disclosed examples require that any one or more specific advantages be present or problems be solved. Features, elements, or components of one example can be combined into other examples herein.

Example 1: A system for approximating papillary muscles of a ventricle of a heart. The system may include a first heart anchor configured to couple to a first papillary muscle of the ventricle; a second heart anchor configured to couple to a second papillary muscle of the ventricle; and a tension member configured to extend within the ventricle and couple the first heart anchor to the second heart anchor and apply a tension to approximate the first papillary muscle and the second papillary muscle, the tension member being configured to expand to accommodate expansion of the ventricle.

Example 2: The system of any example herein, in particular Example 1, wherein the tension member is elastic.

Example 3: The system of any example herein, in particular Example 1 or Example 2, wherein the tension member is configured to expand between a first length at a systolic phase of the ventricle and a second length at a diastolic phase of the ventricle, and apply an elastic force between the first length and the second length.

Example 4: The system of any example herein, in particular Examples 1-3, wherein an outer diameter of at least a portion of the tension member is configured to decrease upon the tension member expanding.

Example 5: The system of any example herein, in particular Examples 1-4, wherein at least a portion of the tension member includes corrugations configured to allow the tension member to expand.

Example 6: The system of any example herein, in particular Example 5, wherein the tension member includes a hose having the corrugations.

Example 7: The system of any example herein, in particular Examples 1-6, wherein at least a portion of the tension member includes a woven body.

Example 8: The system of any example herein, in particular Example 7, wherein the woven body includes a plurality of strips configured to slide with respect to each other to allow the tension member to expand.

Example 9: The system of any example herein, in particular Example 8, wherein the plurality of strips are configured to slide longitudinally to allow the tension member to expand.

Example 10: The system of any example herein, in particular Examples 1-9, wherein at least a portion of the tension member includes a plurality of arms configured to scissor to allow the tension member to expand.

Example 11: The system of any example herein, in particular Example 10, further comprising a spring coupled to the plurality of arms and configured to apply an elastic force to the plurality of arms.

Example 12: The system of any example herein, in particular Examples 1-11, wherein at least a portion of the tension member includes a piston.

Example 13: The system of any example herein, in particular Example 12, wherein the piston includes a spring and a plunger, the spring configured to apply an elastic force to the plunger.

Example 14: The system of any example herein, in particular Examples 10-13, further comprising a stop configured to set a defined limit that the tension member may expand to.

Example 15: The system of any example herein, in particular Example 14, wherein the stop is adjustable to adjust the defined limit.

Example 16: The system of any example herein, in particular Examples 1-15, wherein a portion of the tension member includes a tether that is non-expandable.

Example 17: The system of any example herein, in particular Examples 1-16, wherein at least one of the first heart anchor or the second heart anchor comprises a cuff.

Example 18: The system of any example herein, in particular Examples 1-17, wherein both the first heart anchor and the second heart anchor comprise a cuff.

Example 19: The system of any example herein, in particular Example 17 or Example 18, wherein the cuff includes a ratcheting lock.

Example 20: The system of any example herein, in particular Examples 1-19, wherein at least one of the first heart anchor or the second heart anchor comprises one or more sutures.

Example 21: The system of any example herein, in particular Examples 1-20, further comprising a third heart anchor configured to couple to a third papillary muscle of the ventricle, and wherein the tension member is configured to couple to the third heart anchor.

Example 22: The system of any example herein, in particular Example 21, wherein the tension member comprises a ring.

Example 23: The system of any example herein, in particular Example 22, wherein the tension member includes: a first tether configured to extend radially outward from the ring to the first heart anchor; a second tether configured to extend radially outward from the ring to the second heart anchor; and a third tether configured to extend radially outward from the ring to the third heart anchor.

Example 24: The system of any example herein, in particular Example 22 or Example 23, wherein the ring is configured to expand radially outward to a defined limit.

Example 25: The system of any example herein, in particular Examples 22-24, wherein the ring is configured to provide an elastic force radially inward.

Example 26: A system for approximating papillary muscles of a ventricle of a heart, the system comprising: a first heart anchor configured to be positioned on a first portion of the heart; a second heart anchor configured to be positioned on a second portion of the heart; and a tension member configured to extend within the ventricle and couple the first heart anchor to the second heart anchor and apply a tension to approximate the papillary muscles of the ventricle, the tension member being configured to expand to a defined limit to accommodate expansion of the ventricle.

Example 27: The system of any example herein, in particular Example 26, wherein the tension member is elastic.

Example 28: The system of any example herein, in particular Example 26 or Example 27, wherein the tension member is configured to expand between a first length at a systolic phase of the ventricle and a second length at a diastolic phase of the ventricle, and apply an elastic force between the first length and the second length.

Example 29: The system of any example herein, in particular Examples 26-28, wherein an outer diameter of at least a portion of the tension member is configured to decrease upon the tension member expanding.

Example 30: The system of any example herein, in particular Examples 26-29, wherein at least a portion of the tension member includes corrugations configured to allow the tension member to expand.

Example 31: The system of any example herein, in particular Examples 26-30, wherein at least a portion of the tension member includes a woven body.

Example 32: The system of any example herein, in particular Example 31, wherein the woven body includes a plurality of strips configured to slide with respect to each other to allow the tension member to expand.

Example 33: The system of any example herein, in particular Example 32, wherein the plurality of strips are configured to slide longitudinally to the defined limit.

Example 34: The system of any example herein, in particular Examples 26-33, wherein at least a portion of the tension member includes a plurality of arms configured to scissor to allow the tension member to expand.

Example 35: The system of any example herein, in particular Example 34, further comprising a spring coupled to the plurality of arms and configured to apply an elastic force to the plurality of arms.

Example 36: The system of any example herein, in particular Examples 26-35, wherein at least a portion of the tension member includes a piston.

Example 37: The system of any example herein, in particular Example 36, wherein the piston includes a spring and a plunger, the spring configured to apply an elastic force to the plunger.

Example 38: The system of any example herein, in particular Examples 34-37, further comprising a stop configured to set the defined limit of the tension member.

Example 39: The system of any example herein, in particular Example 38, wherein the stop is adjustable to adjust the defined limit of the tension member.

Example 40: The system of any example herein, in particular Examples 26-39, wherein at least one of the first heart anchor or the second heart anchor comprises a cuff configured to extend around a papillary muscle.

Example 41: The system of any example herein, in particular Examples 26-40, wherein both the first heart anchor and the second heart anchor comprise a cuff configured to extend around a papillary muscle.

Example 42: The system of any example herein, in particular Example 40 or Example 41, wherein the cuff includes a ratcheting lock.

Example 43: The system of any example herein, in particular Examples 26-42, wherein at least one of the first heart anchor or the second heart anchor comprises a pad configured to be positioned on a wall of the ventricle.

Example 44: The system of any example herein, in particular Examples 26-43, wherein both the first heart anchor and the second heart anchor comprise a pad configured to be positioned on a wall of the ventricle.

Example 45: The system of any example herein, in particular Example 43 or Example 44, wherein the pad includes: a ring having two ends and configured to move from a linearized configuration to a ring-shaped configuration; and a cover coupled to the ring and extending inward from the ring in the ring-shaped configuration.

Example 46: The system of any example herein, in particular Examples 26-45, further comprising a third heart anchor configured to couple to a third portion of the heart, and wherein the tension member is configured to couple to the third heart anchor.

Example 47: The system of any example herein, in particular Example 46, wherein the tension member comprises a ring.

Example 48: The system of any example herein, in particular Example 47, wherein the tension member includes: a first tether configured to extend radially outward from the ring to the first heart anchor; a second tether configured to extend radially outward from the ring to the second heart anchor; and a third tether configured to extend radially outward from the ring to the third heart anchor.

Example 49: The system of any example herein, in particular Example 47 or Example 48, wherein the ring is configured to expand radially outward to the defined limit.

Example 50: The system of any example herein, in particular Examples 47-49, wherein the ring is configured to provide an elastic force radially inward.

Example 51: A method for approximating papillary muscles of a ventricle of a heart, the method comprising: deploying a first heart anchor to a first papillary muscle of the ventricle; deploying a second heart anchor to a second papillary muscle of the ventricle; and tensioning a tension member for coupling the first heart anchor to the second heart anchor to approximate the papillary muscles of the ventricle, the tension member extending within the ventricle and configured to expand to accommodate expansion of the ventricle.

Example 52: The method of any example herein, in particular Example 51, wherein the tension member is configured to expand between a first length at a systolic phase of the ventricle and a second length at a diastolic phase of the ventricle, and apply an elastic force between the first length and the second length.

Example 53: The method of any example herein, in particular Example 51 or Example 52, wherein at least a portion of the tension member includes corrugations configured to allow the tension member to expand.

Example 54: The method of any example herein, in particular Examples 51-53, wherein at least a portion of the tension member includes a woven body.

Example 55: The method of any example herein, in particular Examples 51-54, wherein at least a portion of the tension member includes a plurality of arms configured to scissor to allow the tension member to expand.

Example 56: The method of any example herein, in particular Examples 51-55, wherein at least a portion of the tension member includes a piston.

Example 57: The method of any example herein, in particular Example 55 or Example 56, further comprising a stop configured to set a defined limit that the tension member may expand to.

Example 58: The method of any example herein, in particular Example 57, wherein the stop is adjustable to adjust the defined limit.

Example 59: The method of any example herein, in particular Examples 51-58, wherein at least one of the first heart anchor or the second heart anchor comprises a cuff.

Example 60: The method of any example herein, in particular Examples 51-59, wherein at least one of the first heart anchor or the second heart anchor comprises one or more sutures.

Example 61: The method of any example herein, in particular Examples 51-60, further comprising: deploying a third heart anchor to a third papillary muscle of the ventricle; and wherein the tension member is configured to couple to the third heart anchor.

Example 62: The method of any example herein, in particular Example 61, wherein the tension member comprises a ring.

Example 63: The method of any example herein, in particular Example 62, wherein the tension member includes: a first tether configured to extend radially outward from the ring to the first heart anchor; a second tether configured to extend radially outward from the ring to the second heart anchor; and a third tether configured to extend radially outward from the ring to the third heart anchor.

Example 64: The method of any example herein, in particular Example 62 or Example 63, wherein the ring is configured to expand radially outward to a defined limit.

Example 65: The method of any example herein, in particular Examples 62-64, wherein the ring is configured to provide an elastic force radially inward.

Example 66: A method for approximating papillary muscles of a ventricle of a heart, the method comprising: deploying a first heart anchor to a first portion of the heart; deploying a second heart anchor to a second portion of the heart; and tensioning a tension member for coupling the first heart anchor to the second heart anchor to approximate the papillary muscles of the ventricle, the tension member extending within the ventricle and configured to expand to a defined limit to accommodate expansion of the ventricle.

Example 67: The method of any example herein, in particular Example 66, wherein the tension member is configured to expand between a first length at a systolic phase of the ventricle and a second length at a diastolic phase of the ventricle, and apply an elastic force between the first length and the second length.

Example 68: The method of any example herein, in particular Example 66 or Example 67, wherein at least a portion of the tension member includes corrugations configured to allow the tension member to expand.

Example 69: The method of any example herein, in particular Examples 66-68, wherein at least a portion of the tension member includes a woven body.

Example 70: The method of any example herein, in particular Examples 66-69, wherein at least a portion of the tension member includes a plurality of arms configured to scissor to allow the tension member to expand.

Example 71: The method of any example herein, in particular Examples 66-70, wherein at least a portion of the tension member includes a piston.

Example 72: The method of any example herein, in particular Example 70 or Example 71, further comprising a stop configured to set the defined limit of the tension member.

Example 73: The method of any example herein, in particular Example 72, wherein the stop is adjustable to adjust the defined limit of the tension member.

Example 74: The method of any example herein, in particular Examples 66-73, wherein at least one of the first heart anchor or the second heart anchor comprises a cuff configured to extend around a papillary muscle.

Example 75: The method of any example herein, in particular Examples 66-74, wherein at least one of the first heart anchor or the second heart anchor comprises a pad configured to be positioned on a wall of the ventricle.

Example 76: The method of any example herein, in particular Examples 66-75, further comprising: deploying a third heart anchor to a third portion of the heart; and wherein the tension member is configured to couple to the third heart anchor.

Example 77: The method of any example herein, in particular Example 76, wherein the tension member comprises a ring.

Example 78: The method of any example herein, in particular Example 77, wherein the tension member includes: a first tether configured to extend radially outward from the ring to the first heart anchor; a second tether configured to extend radially outward from the ring to the second heart anchor; and a third tether configured to extend radially outward from the ring to the third heart anchor.

Example 79: The method of any example herein, in particular Example 77 or Example 78, wherein the ring is configured to expand radially outward to the defined limit.

Example 80: The method of any example herein, in particular Examples 77-79, wherein the ring is configured to provide an elastic force radially inward.

Any of the features of any of the examples, including but not limited to any of the first through eightieth examples referred to above, is applicable to all other aspects and examples identified herein, including but not limited to any examples of any of the first through eightieth examples referred to above. Moreover, any of the features of an example of the various examples, including but not limited to any examples of any of the first through eightieth examples referred to above, is independently combinable, partly or wholly with other examples described herein in any way, e.g., one, two, or three or more examples may be combinable in whole or in part. Further, any of the features of the various examples, including but not limited to any examples of any of the first through eightieth examples referred to above, may be made optional to other examples. Any example of a method can be performed by a system or apparatus of another example, and any aspect or example of a system or apparatus can be configured to perform a method of another aspect or example, including but not limited to any examples of any of the first through eightieth examples referred to above.

In closing, it is to be understood that although aspects of the present specification are highlighted by referring to specific examples, one skilled in the art will readily appreciate that these disclosed examples are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular examples only, and is not intended to limit the scope of systems, apparatuses, and methods as disclosed herein, which is defined solely by the claims. Accordingly, the systems, apparatuses, and methods are not limited to that precisely as shown and described.

Certain examples of systems, apparatuses, and methods are described herein, including the best mode known to the inventors for carrying out the same. Of course, variations on these described examples will become apparent to those of ordinary skill in the art upon reading the foregoing description. The inventor expects skilled artisans to employ such variations as appropriate, and the inventors intend for the systems, apparatuses, and methods to be practiced otherwise than specifically described herein. Accordingly, the systems, apparatuses, and methods include all modifications and equivalents of the subject matter recited in the claims appended hereto as permitted by applicable law. Moreover, any combination of the above-described examples in all possible variations thereof is encompassed by the systems, apparatuses, and methods unless otherwise indicated herein or otherwise clearly contradicted by context.

Groupings of alternative examples, elements, or steps of the systems, apparatuses, and methods are not to be construed as limitations. Each group member may be referred to and claimed individually or in any combination with other group members disclosed herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses an approximation that may vary, yet is capable of performing the desired operation or process discussed herein.

The terms “a,” “an,” “the” and similar referents used in the context of describing the systems, apparatuses, and methods (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the systems, apparatuses, and methods and does not pose a limitation on the scope of the systems, apparatuses, and methods otherwise claimed. No language in the present specification should be construed as indicating any non-claimed element essential to the practice of the systems, apparatuses, and methods.

All patents, patent publications, and other publications referenced and identified in the present specification are individually and expressly incorporated herein by reference in their entirety for the purpose of describing and disclosing, for example, the compositions and methodologies described in such publications that might be used in connection with the systems, apparatuses, and methods. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

What is claimed is:

1. A system for approximating papillary muscles of a ventricle of a heart, the system comprising:
a first heart anchor configured to couple to a first papillary muscle of the ventricle;
a second heart anchor configured to couple to a second papillary muscle of the ventricle; and
a tension member configured to extend within the ventricle and couple the first heart anchor to the second heart anchor and apply a tension to approximate the first papillary muscle and the second papillary muscle, the tension member being configured to expand to accommodate expansion of the ventricle.

2. The system of claim 1, wherein the tension member is elastic.

3. The system of claim 1, wherein the tension member is configured to expand between a first length at a systolic phase of the ventricle and a second length at a diastolic phase of the ventricle, and apply an elastic force between the first length and the second length.

4. The system of claim 1, wherein an outer diameter of at least a portion of the tension member is configured to decrease upon the tension member expanding.

5. The system of claim 1, wherein at least a portion of the tension member includes corrugations configured to allow the tension member to expand.

6. The system of claim 5, wherein the tension member includes a hose having the corrugations.

7. The system of claim 1, wherein at least a portion of the tension member includes a woven body.

8. The system of claim 7, wherein the woven body includes a plurality of strips configured to slide with respect to each other to allow the tension member to expand.

9. The system of claim 8, wherein the plurality of strips are configured to slide longitudinally to allow the tension member to expand.

10. The system of claim 1, wherein at least a portion of the tension member includes a plurality of arms configured to scissor to allow the tension member to expand.

11. The system of claim 10, further comprising a spring coupled to the plurality of arms and configured to apply an elastic force to the plurality of arms.

12. The system of claim 1, wherein at least a portion of the tension member includes a piston.

13. The system of claim 12, wherein the piston includes a spring and a plunger, the spring configured to apply an elastic force to the plunger.

14. The system of claim 10, further comprising a stop configured to set a defined limit that the tension member may expand to.

15. The system of claim 14, wherein the stop is adjustable to adjust the defined limit.

16. The system of claim 1, wherein a portion of the tension member includes a tether that is non-expandable.

17. The system of claim 1, wherein at least one of the first heart anchor or the second heart anchor comprises a cuff.

18. The system of claim 1, wherein both the first heart anchor and the second heart anchor comprise a cuff.

19. The system of claim 17, wherein the cuff includes a ratcheting lock.

20. The system of claim 1, wherein at least one of the first heart anchor or the second heart anchor comprises one or more sutures.
